(19) United States
(12) Patent Application Publication
(10) Pub. No.: US 2005/0048045 A1
(43) Pub. Date: Mar. 3, 2005

(54) Regulation of urokinase receptor expression by phosphoglycerate kinase

(75) Inventors: Sreerama Shetty, Tyler, TX (US); Steven Idell, Tyler, TX (US)

Correspondence Address: FULBRIGHT & JAWORSKI L.L.P., 600 CONGRESS AVE., SUITE 2400, AUSTIN, TX, 78701, US

(73) Assignee: Board of Regents, The University of Texas System

(21) Appl. No.: 10/884,195

(22) Filed: Jul. 2, 2004

Related U.S. Application Data
(60) Provisional application No. 60/484,562, filed on Jul. 2, 2003.
(60) Provisional application No. 60/489,018, filed on Jul. 22, 2003.

(51) Int. Cl. A61K 38/48

(52) U.S. Cl. 424/94.5

(57) ABSTRACT

The present invention provides methods for treating inflammatory diseases, neoplastic diseases, wound healing and preventing scarring by administering a therapeutically effective amount of phosphoglycerate phosphokinase (PGK) peptide, polypeptide, protein, mutant or mimetic to a subject. The invention also relates to methods of screening for compounds for modulation of uPAR expression or activity. The present invention further provides coding sequences of phosphoglycerate kinase peptides, polypeptides, or proteins, or mutants, or mimetics thereof as a gene therapy for inflammatory diseases, cancer, wound healing, or tissue scarring.

The present application claims the benefit of U.S. Provisional Application Serial No. 60/484,562, filed on Jul. 2, 2003 and U.S. Provisional Application Serial No. 60/489,018 filed on Jul. 22, 2003. The entire text of the above-referenced disclosures are specifically incorporated herein by reference without disclaimer.

The government may own rights in the present invention pursuant to grant number RO1 62453 from the National Institutes of Health.

### BACKGROUND OF THE INVENTION

1. Field of the Invention

The present invention relates generally to the field of molecular pharmacology. More particularly, the present invention concerns compositions and methods for the treatment of inflammatory and neoplastic diseases.

2. Description of Related Art

Dissolution of extracellular matrix proteins by serine proteinases and metalloproteinases has been implicated in the pathogenesis of several diseases including lung injury and lung neoplasia (Mazar et al., 1999; Dvorak, 1986; Dvorak et al., 1983). These proteinases influence inflammatory cell traffic or cancer cell invasiveness via the degradation of basement membranes and extracellular matrix (Vassalli et aL., 1991; Dano et al., 1985; Liotta et al., 1991; Mignatti and Rifkin, 1993). Plasmin, a serine protease, is generated via the action of plasminogen activators such as urokinase (uPA) or tissue plasminogen activator (tPA). Plasmin can influence tissue remodeling either directly or through activation of latent collagenases. uPA plays a pivotal role in extravascular proteolysis and is implicated in stromal remodeling in acute and chronic lung injury (Idell, 1994; Idell, 1995; Chapman et al., 1988) and in tumor metastasis (Mignatti and Rifkin, 1993). During the last decade, evidence for the involvement of the uPA system in lung injury and repair or lung neoplasia (Mazar et al., 1999) has steadily increased and it is now indicated that uPA-dependent plasminogen activation is central to these processes.

Many biological activities of uPA depend upon association with the urokinase receptor (uPAR), which plays a central role in localized uPA-mediated plasminogen activation. Increased expression of uPA or uPAR has, for example, been inversely correlated with prognosis in lung cancer (Pedersen et al., 1994a; Pedersen et al., 1994b). Interaction of uPA with its cellular receptor also has several unique properties that promote cellular signaling, which can thereby influence the course of lung inflammation or cancer. Better understanding of the specific pathways that regulate uPAR expression is therefore germane to the pathogenesis of lung injury or the spread of lung neoplasms.

Expression of uPAR at the cell surface is implicated in a variety of cellular responses involved in the pathogenesis of inflammation as well as cancer. The interaction between uPA and uPAR at the cell surface appears to influence neoplastic tissue remodeling, tumor cell invasion, adhesion and proliferation (Idell, 1995; Idell, 1994; Vasalli et al., 1991; Mazar et al., 1999; Ghiso et al., 1999; Shetty et al., 1995a; Shetty et al., 1995b; Waltz et al., 1993; Bhat et al., 1999; Xing et al., 1997). In addition, the binding of uPA to uPAR mediates cellular proteolysis and cellular proliferation in several cell-types including nonmalignant and malignant lung epithelial cells and mesothelioma cells (Idell et al., 1994; Shetty and Idell, 1998; Shetty et al., 1995a; Shetty et al., 1995b; Bhat et al., 1999).

Synthesis of uPAR can be regulated transciptionally or post-transcriptionally. The increased uPAR mRNA stability in lung-derived epithelial and mesothelioma cells correlates with increased uPAR mRNA and cell surface expression of uPAR. Lymphocyte engagement, for example, also stabilizes uPAR mRNA, a process that involves AU-rich sequences present in the uPAR 3′ untranslated region (3′ UTR) (Wang et al., 1998). The steady-state of any mRNA reflects its synthesis as well as lability. The expression of a number of proteins is regulated by specific and rapid decay of their transcripts. Among the various mechanisms by which different cell types regulate mRNA stability, control of mRNA decay is a potentially important process determining the level of gene expression in eukaryotic cells. Synthesis of uPAR is regulated by a variety of hormones, growth factors and cytokines either at the transcriptional or posttrancriptional level (Blasi et al., 1987a; Lund et al., 1995a; Lund et al., 1991a; Shetty et al., 1997; Wang et al., 1994; Wang et al., 1998; Pepper et al., 1992; Sprengers and Kluft, 1987). Both uPA and uPAR, as well as PAI-1 and -2 are expressed by lung epithelial cells (Idell et al., 1994; Shetty and Idell, 2000a; Shetty and Idell, 2000b), and it is now clear that posttranscriptional regulation contributes to the regulation of uPA, uPAR and PAI-1 by these cells (Shetty et al., 1997; Shetty and Idell 2000a; Shetty and Idell 2000b; Shetty and Idell 1999; Shetty and Idell, 1998a; Shetty and Idell, 1998b). These mechanisms involve cis-trans interactions that involve the 3′ untranslated region (UTR) and, in some cases the coding region of the mRNA. Interaction of uPA with its receptor uPAR, have been shown to control a variety of cellular functions including epithelial cell adhesion, signaling and mitogenesis. This interaction has also been shown to promote lung inflammation and/or cancer.

Unfortunately, despite the above, the identity of regulatory proteins or substances that can regulate the expression of either uPA or uPAR and/or the interaction of uPA and uPAR has largely been unknown. There is, therefore, a great need in the art for the identification of substances interacting with uPAR in particular. Identification of such substances could provide new therapeutics that prevent inflammatory diseases and/or cancer by disrupting or modulating uPAR activity, an interaction thus far difficult to achieve.

### SUMMARY OF THE INVENTION

The present invention relates to the purification and identification of phosphoglycerate kinase (PGK), a 50 kDa cytoplasmic protein that specifically interacts with the urokinase receptor (uPAR) coding region mRNA. The inventors importantly found that overexpression of this protein in lung carcinoma cells resulted in a decrease of uPAR activity and expression. Therefore, the present invention provides PGK peptides, polypeptides or protein and/or mutants or mimetic thereof and the use of such compounds in the treatment of diseases and conditions that involve uPAR.

Thus, the present invention provides a method of treating an inflammatory and/or neoplastic disease comprising administering to a subject a therapeutically effective amount of a phosphoglycerate kinase peptide, polypeptide, protein, or a mutant or mimetic thereof; and inhibiting urokinase receptor activity or expression in the subject. Inflammatory diseases that can be treated by the present invention include inflammatory diseases of the lung, thyroid, larynx, bladder, colon, esophagus, gastrointestine, gum, nasopharynx, or skin, but are not limited to such. It is further contemplated that the inflammatory disease may be psoriasis, atopic dermatitis, nonspecific dermatitis, allergic contact dermatitis, primary irritant contact dermatitis, cutaneous basal cell carcinoma, cutaneous planocellular carcinoma, lameliar ichthyosis, epidemolytic keratosis, solar induced precancerous keratosis, benign keratosis, seborrheic dermatitis, keloids, dermatomyositis, angiogenesis-related skin disorders, and erythroderma, but are not limited to such. Tissue fibrosis, such as occurs in pulmonary fibrosis or fibrotic repair in other organs can also be treated by the methods described herein.

In some embodiments of the invention, a method to prevent sepsis or inflammation after an injury such as an immune tissue injury is provided. A tissue injury may include an injury that results from exposure to environmental agents, but is not limited to such.

Neoplastic disease to be treated may be a cancer such as a premalignant, malignant, or metastastic cancer. In further embodiments of the invention the cancer may be a cancer of the lung, breast, head and neck, bladder, bone, bone marrow, brain, colon, esophagus, gastrointestine, gum, kidney, liver, nasopharynx, ovary, prostate, skin, stomach, testis, tongue, or uterus.

The phosphoglycerate kinase peptide, polypeptide, protein, or mutant or mimetic thereof may be administered to a subject intravenously, intralesionally, percutaneously, subcutaneously, or by an aerosol. The PGK peptide, polypeptide or protein or mutant thereof or mimetic thereof may comprise a sequence of SEQ ID NO: 2.

In particular embodiments, the phosphogylcerate kinase peptide, polypeptide, or protein, or mutant or mimetic thereof reduces, decrease or inhibits urokinase receptor (uPAR) activity or expression.

In further embodiments, the present invention contemplated delivering an expression construct comprising a nucleic acid encoding a phosphogylcerate kinase peptide, polypeptide, or protein, or mutant or mimetic to a subject. Such an expression construct may be a viral vector including an adenoviral vector, an adeno-associated viral vector, a herpesviral vector, a retroviral vector, a lentiviral vector, a vaccinia viral vector, or a polyoma vector. The expression construct may be delivered, intravenously, intralesionally, percutaneously, subcutaneously, or by an aerosol.

In further embodiments the present invention comprises phosphoglycerate kinase gene therapy.

In another particular embodiment, the present invention provides a method of screening for a candidate substance for modulation of uPAR expression or activity comprising: (a) providing a uPAR in a cell or cell-free assay mixture; (b) contacting the uPAR with the candidate substance; and (c) measuring the uPAR activity or expression, wherein a decrease in the uPAR activity or expression in the presence of the candidate modulator as compared to the uPAR activity or expression in a cell or cell-free assay mixture not exposed to the candidate modulator indicates that the candidate PGK substance has the ability to downregulate or inhibit the expression or activity of uPAR. The cell may be an inflammatory disease cell or a neoplastic disease cell located in a subject such as a mammal. In some embodiments of the invention, the mammal may be a human. In some aspects of the invention, the cell is in vitro. In certain aspects of the invention, the method may further comprise the step of (d) manufacturing the candidate substance. The method may still further comprise administering the candidate substance that is manufactured to a subject, for example, a human.

In further embodiments of the invention, measuring the uPAR activity or expression may comprises Northern blotting or Western blotting.

In yet another particular embodiment, the present invention provides a method for preventing or decreasing scarring in a subject comprising administering to the subject a therapeutically effective amount of a phosphoglycerate kinase peptide, polypeptide, protein, or a mutant or mimetic thereof; and inhibiting urokinase receptor (uPAR) activity or expression in the subject.

In still yet another particular embodiment, the present invention provides a method for promoting wound healing in a subject comprising administering to the subject a therapeutically effective amount of a phosphoglycerate kinase peptide, polypeptide, protein, or a mutant or mimetic thereof; and inhibiting urokinase receptor (uPAR) activity or expression in the subject.

It still yet another embodiment, the present invention provides a pharmaceutical composition comprising a phosphoglycerate kinase peptide, polypeptide, protein, or a mutant, or a mimetic thereof.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1D. Purification and analysis of uPAR mRNA binding proteins in Beas2B cells. FIG. 1A—Silver staining of purified preparation from Beas2B cells (lane 1). FIG. 1B—gel mobility shift assays (lane 1, free probe; lane 2, purified protein). FIG. 1C—UV cross-linking assays (lane 1, free probe; lane 2, pure protein). FIG. 1D Northwestern blot analysis (lane 1).

FIGS. 2A-2B. Urokinase receptor mRNA binding activity of recombinant PGK by gel mobility shift assay. FIG. 2A—uPAR mRNA binding activity demonstrated by gel mobility shift assay. Five microgram of each protein was used. Protein eluate of cytosolic extracts of non-tranfected H157 cells (H157) or H157 cells transfected with Destination 40 vector alone (D40) or PGK cDNA in Destination vector 40 (PGK) purified on Nickel column. Yeast PGK (PGK-Y) or recombinant PGK purified from H157 cells in the presence of a 200-fold molar excess of unlabeled par CD RNA (PG.-C). FIG. 2B—PG. protein purified from prokaryotic expression vector using Nickel column. IPTG induced BL21 bacterial lysates eluted from the Nickel column (BL21). Bacterial lysates from IPTG induced cell lysates of BL21 cells transfected with PGK cDNA (PGK), PGK purified from BL21 in the presence of 200-fold molar excess of unlabeled uPAR mRNA (PGK-C). Fp=free probe.

FIGS. 3A-3C. Assessment of uPAR mRNA binding activity and molecular weight of PGK. The proteins prepared as described in FIG. 2, were separated on SDS-PAGE, transferred to a nitrocellulose membrane. The membranes were later developed with uPAR mRNA coding region transcript and autoradiography. FIG. 3A—Protein eluate of cytosolic extracts of untransfected H157 cells (H157) or H157 cells transfected with Destination 40 vector alone (D40) or PGK cDNA in Destination vector 40 (PGK) purified on a Nickel column. FIG. 3B—Yeast PGK (PGK-Y) protein purified from a prokaryotic expression vector using Nickel column. IPTG induced BL21 bacterial lysates eluted from the Nickel column (BL21). Bacterial lysates from IPTG induced cell lysate of BL21 cells transfected with PGK cDNA (PGK). FIG. 3C—The corresponding membrane (FIG. 3A) stripped and developed using anti-V5 monoclonal antibody.

FIGS. 4A-4B. Inhibition of uPAR expression by PGK in H157 cells. FIG. 4A—Control H157 cells or stable H157 cells transfected with empty vector or PGK cDNA were grown to confluence. The membrane proteins isolated from these cells were separated on 8% SDS-PAGE and electroblotted to nitrocellulose membranes. The membranes were subjected to Western blotting using a urokinase receptor monoclonal antibody. FIG. 4B—Urokinase receptor mRNA expression in PGK transfected H157 cells. H157 cell lines as mentioned in FIG. 4A were grown to confluence in RPMI 1640 media with or without G418. Total RNA was isolated and uPAR mRNA was measured by Northern blot using ³²P-labeled uPAR cDNA. The data illustrated is representative of four independent studies.

FIGS. 5A-5B. Effect of PGK on the rate of transcription and decay of uPAR mRNA in H157 Cells. FIG. 5A—Nuclei isolated from H157 cell lines as described above were subjected to the transcription reaction in the presence of ³²P UTP at 30° C. for 30 min. ³²P-labeled nuclear RNA was hybridized with uPAR cDNA immobilized on nitrocellulose membrane. β-actin and PUC 18 cDNAs were used as positive and negative loading controls respectively. FIG. 5B—Effect of PGK on uPAR mRNA stability. Ongoing transcription by H157 cells grown to confluence was inhibited by treating the cells with DRB for 0-24 h in the same medium. Total RNA was isolated and uPAR was analyzed by Northern blot.

FIGS. 6A-6B. Inhibition of H157 cells ³H-thymidine uptake by PGK. FIG. 6A—The H157 cell lines were serum starved overnight and treated with 3H-thymidine for 6h. The cells were washed and the rate of DNA synthesis was measured by measuring 3H-thymidine incorporation. FIG. 6B—Effect of PGK expression invasion/migration ratio of H157 cells. The cells grown to confluence were transferred to the upper chamber of Transwell plates for assessment in the invasion/migration assay. The cells were incubated for 72 h at 37° C., the number of cells present in the upper and the lower chambers were counted and the invasion/migration ratios (IMR) were calculated based on the total number of cells present in both the upper and lower chambers.

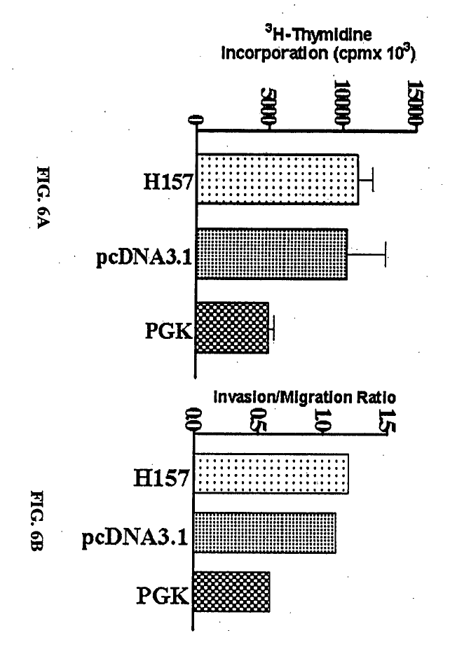

### DESCRIPTION OF ILLUSTRATIVE EMBODIMENTS

The inventors purified a cytoplasmic protein that specifically interacts with the uPAR coding region mRNA and identified it as the 50 kDa protein phosphoglycerate kinase (PGK). The cDNA was cloned and the recombinant protein expressed in bacteria and eukaryotic cells. Both these preparations specifically bound to the urokinase receptor (uPAR) mRNA coding region. Overexpression of PGK protein in squamous cell carcinoma reduced cell surface uPAR expression as well as uPAR mediated cellular functions by a posttranscriptional mechanism. Immunochemical analyses confirmed that PGK is the uPAR mRNA binding protein (uPAR mRNABp) (Shetty et al., 1997).

The studies described herein demonstrate that PGK regulates uPAR mRNA expression at the posttranscriptional level in cultured H157 cells. This pathway involves interaction of PGK with the uPAR mRNA coding region and regulates expression of uPAR at the cell surface. This mechanism represents a pathway by which uPAR-dependent responses of the lung epithelium may be controlled in the context of lung injury and repair, neoplastic transformation or in the growth and spread of lung neoplasms.

The interaction between uPA and uPAR at the cancer cell is a critical determinant of the pathogenesis of neoplastic growth and metastasis, mediating tissue remodeling, tumor cell invasion, adhesion and proliferation. Many biological activities of uPA depend on association with its receptor, uPAR plays a central role in localizing uPA-mediated plasminogen activation and cellular signaling. Regulation of this receptor thus allows one to influence the broad range of uPA activity and its effect on cellular pathophysiology. Posttranscriptional regulation of uPAR can, therefore, influence a wide range of pathophysiologic responses germane to lung inflammation as well as cancer.

The studies described herein demonstrate the role of PGK in the regulatory mechanism that controls uPAR expression at the posttranscriptional level, and confirm the involvement of PGK in this process. PGK specifically interacts with uPAR mRNA, and overexpression of this protein down regulates uPAR expression as well uPAR-mediated cellular proliferation and migration of H157 cells. The results provide an example of a functional and biochemical chimerism, namely mRNA binding coupled with metabolic enzyme activity.

The inventors' discovery of the relationship between PGK and uPA as described herein enables those of skill to treat a wide variety of diseases and conditions using techniques and methods described in detail below and known to those of skill. Additionally, the determination of this relationship enables those of skill to ascertain appropriate treatment agents, dosages, and treatment regimes as described herein and known to those of skill.

I. Phosphoglycerate Kinase

In humans and other mammals, the glycolytic enzyme phosphoglycerate kinase (PGK; ATP:3-phospho-D-glycerate 1-phosphotransferase) is a metabolic enzyme functioning in the Embden-Meyerhof pathway that converts glucose (or fructose) to pyruvate. This enzyme is expressed in various tissues, including red blood cells, and is encoded by a single structural gene on the X-chromosome, band q13 in humans (Boer et al., 1987). This enzyme is encoded by two genes, PGK1 and PGK2 (Boer et al., 1987; McCarrey et al., 1987; Michelson et al., 1985). The mature enzyme consists of 416 amino acid residues with acetyl-serine at the NH-terminal and isoleucine at the COOH-terminal, and the monomeric enzyme (MW ˜48 kD) is catalytically active (McCarrey et al., 1987). The human PGK-1 gene consists of 11 exons and 10 introns encompassing a region approximately 23 kilobases (kb) in length.

Although the two genes produce proteins that are both structurally and functionally similar (McCarrey et al., 1996), they differ markedly in the manner in which their expression is regulated (McCarrey et al., 1996; McCarrey, 1987; McCarrey, 1990; McCarrey et al., 1992; McCarrey, 1994). PGK1 is X-chromosome-linked and is ubiquitously expressed at relatively low levels in all somatic cells. PGK1 is also expressed in oogenic cells, and premeiotic spermatogenic cells (McCarrey et al., 1992; VandeBerg, 1985). PGK2 is autosomal and is expressed in a tissue- and cell-type specific manner at relatively high levels during spermatogenesis, specifically in meiotic and postmeiotic spermatogenic cells (McCarrey et al., 1992; VandeBerg and Cooper, 1976; Kramer and Erickson, 1981).

Several lines of evidence suggest that the PGK2 gene arose as a processed duplication of the PGK1 gene through RNA-mediated retroposition early during mammalian evolution (McCarrey and Thomas, 1987; McCarrey, 1994). Additional data suggest that the original PGK2 retroposon carried a copy of the progenitor “PGK1-like” promoter that directed ubiquitous expression, and that this promoter subsequently evolved a tissue-specific regulatory function so that it now directs testis-specific transcription (McCarrey, 1990; McCarrey, 1994). In mammalian spermatogenic cells, transcriptional repression of the Pgk-1 gene occurs as a result of X chromosome inactivation (XCI) during prophase of meiosis I (McCarrey et al., 1992a; McCarrey et al., 1992b). At the same time, transcription of the autosomal Pgk-2 gene is initiated. Conservation of a duplicate PGK gene expressed uniquely in meiotic and postmeiotic spermatogenic cells proved to be advantageous because it provided an alternate source of PGK to compensate for repressed expression of the PGK1 gene resulting from X-chromosome inactivation during spermatogenesis (McCarrey et al., 1992; McCarrey, 1994).

Transcription of the PGK2 gene is controlled by regulatory sequences located in the 5′ flanking region of the gene (McCarrey, 1987; Robinson et al., 1989; Gebara and McCarrey, 1992; Berg et al., 1993; Kumari et al., 1996). This region includes both core promoter sequences and tissue-specific enhancer sequences. Core promoter function has been demonstrated in the first 188 base pairs (bp) upstream from the translational start site in the human PGK2 gene, including a 70-bp 5′ untranslated region (Berg et al., 1993). This core promoter contains a CAAT-box and a GC-box upstream from the single transcriptional start site, but lacks a TATA-box (McCarrey, 1987). It has been shown that CAAT- and GC-boxes act as binding sites for the ubiquitous transcription factors, CTF-1 and Sp1, respectively (Jones et al., 1985; Kadonaga et al., 1987). Similar results were noted for PGK2 promoter Berg et al. (1993).

II. Phosphoglycerate Kinase Proteins, Polypeptides, and Peptides

The present invention employs phosphoglycerate kinase peptide, polypeptide, protein, or a mutant or mimetic thereof for treating inflammatory and/or neoplastic diseases, and for promoting wound healing, and to prevent or decrease scarring.

The present invention provides purified, and in preferred embodiments, substantially purified mammalian phosphoglycerate kinase proteins, polypeptides, or peptides. The term “purified mammalian phosphoglycerate kinase proteins, polypeptides, or peptides” as used herein, is intended to refer to an phosphoglycerate kinase proteinaceous composition, isolatable from mammalian cells or recombinant host cells, wherein the phosphoglycerate kinase protein, polypeptide, or peptide is purified to any degree relative to its naturally-obtainable state, i.e., relative to its purity within a cellular extract. A purified phosphoglycerate kinase protein, polypeptide, or peptide therefore also refers to a wild-type or mutant phosphoglycerate kinase protein, polypeptide, or peptide free from the environment in which it naturally occurs.

The phosphoglycerate kinase proteins may be full length proteins, such as being 416 amino acids in length. The phosphoglycerate kinase proteins, polypeptides and peptides may also be less then full length proteins, such as individual polypeptide, domains, regions or even epitopic peptides. Where less than full length phosphoglycerate kinase proteins are concerned the most preferred will be those containing predicted immunogenic sites and those containing the functional domains identified herein.

Encompassed by the invention are proteinaceous segments of relatively small peptides, such as, for example, peptides of from about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 35, about 40, about 45, to about 50 amino acids in length, and more preferably, of from about 15 to about 30 amino acids in length; as set forth in SEQ ID NO:2 and also larger polypeptides of from about 51, about 52, about 53, about 54, about 55, about 56, about 57, about 58, about 59, about 60, about 65, about 70, about 75, about 80, about 85, about 90, about 95, about 100, about 110, about 120, about 130, about 140, about 150, about 160, about 170, about 180, about 190, about 200, abou 220, about 240, about 260, about 280, about 300, about 320, about 340, about 360, about 380, about 400, about 420, up to and including proteins corresponding to the full-length sequences set forth in SEQ ID NO:2.

Generally, “purified” will refer to an phosphoglycerate kinase protein, polypeptide, or peptide composition that has been subjected to fractionation to remove various non-phosphoglycerate kinase protein, polypeptide, or peptide, and which composition substantially retains its phosphoglycerate kinase activity, as may be assessed, assays described herein or would be known to one of skill in the art.

Where the term “substantially purified” is used, this will refer to a composition in which the phosphoglycerate kinase protein, polypeptide, or peptide forms the major component of the composition, such as constituting about 50% of the proteinaceous molecules in the composition or more. In preferred embodiments, a substantially purified proteinaceous molecule will constitute more than 60%, 70%, 80%, 90%, 95%, 99% or even more of the proteinaceous molecules in the composition.

A peptide, polypeptide or protein that is “purified to homogeneity,” as applied to the present invention, means that the peptide, polypeptide or protein has a level of purity where the peptide, polypeptide or protein is substantially free from other proteins and biological components. For example, a purified peptide, polypeptide or protein will often be sufficiently free of other protein components so that degradative sequencing may be performed successfully.

Various methods for quantifying the degree of purification of phosphoglycerate kinase proteins, polypeptides, or peptides will be known to those of skill in the art in light of the present disclosure. These include, for example, determining the specific proteinaceous molecule's activity of a fraction, or assessing the number of proteins, polypeptides and peptides within a fraction by gel electrophoresis. Assessing the number of proteinaceous molecules within a fraction by SDS/PAGE analysis will often be preferred in the context of the present invention as this is straightforward.

To purify an phosphoglycerate kinase protein, polypeptide, or peptide a natural or recombinant composition comprising at least some phosphoglycerate kinase proteins, polypeptides, or peptides will be subjected to fractionation to remove various non-phosphoglycerate kinase components from the composition. In addition to those techniques described in detail herein below, various other techniques suitable for use in proteinaceous molecule purification will be well known to those of skill in the art. These include, for example, precipitation with ammonium sulfate, PEG, antibodies and the like or by heat denaturation, followed by centrifugation; chromatography steps such as ion exchange, gel filtration, reverse phase, hydroxylapatite, lectin affinity and other affinity chromatography steps; isoelectric focusing; gel electrophoresis; and combinations of such and other techniques. Another example is the purification of an phosphoglycerate kinase fusion protein using a specific binding partner. Such purification methods are routine in the art.

The exemplary purification methods disclosed herein represent exemplary methods to prepare a substantially purified phosphoglycerate kinase protein, polypeptide, or polypeptide. These methods are preferred as they result in the substantial purification of the phosphoglycerate kinase protein, polypeptide or peptide in yields sufficient for further characterization and use. However, given the nucleic acid and proteinaceous molecules provided by the present invention, any purification method can now be employed.

Although preferred for use in certain embodiments, there is no general requirement that the phosphoglycerate kinase protein, polypeptide, or peptide always be provided in their most purified state. Indeed, it is contemplated that less substantially purified phosphoglycerate kinase protein, polypeptide or peptide, which are nonetheless enriched in phosphoglycerate kinase proteinaceous compositions, relative to the natural state, will have. utility in certain embodiments. These include, for example, antibody generation where subsequent screening assays using purified phosphoglycerate kinase proteinaceous molecules are conducted.

Methods exhibiting a lower degree of relative purification may have advantages in total recovery of proteinaceous molecule product, or in maintaining the activity of an expressed proteinaceous molecule. Inactive products also have utility in certain embodiments, such as, e.g., in antibody generation.

III. Mutagenesis and Peptidomimetics

It will also be understood that this invention is not limited to the particular amino acid sequences of SEQ ID NO:2. Recombinant vectors and isolated nucleic acid segments may therefore variously include these coding regions themselves, coding regions bearing selected alterations or modifications in the basic coding region, or they may encode larger polypeptides that nevertheless include such coding regions or may encode biologically functional equivalent proteins, polypeptides or peptides that have variant amino acids sequences.

Nucleic acid segments of the present invention may encompass biologically functional equivalent phosphoglycerate kinase proteins, polypeptides, and peptides. Such sequences may arise as a consequence of codon redundancy and functional equivalency that are known to occur naturally within nucleic acid sequences and the proteinaceous compositions thus encoded. Alternatively, functionally equivalent proteins, polypeptides or peptides may be created via the application of recombinant nucleic acid technology, in which changes in the protein, polypeptide or peptide structure may be engineered, based on considerations of the properties of the amino acids being exchanged. Changes designed by man may be introduced, for example, through the application of site-directed mutagenesis techniques as discussed herein below, e.g., to introduce improvements to the antigenicity of the proteinaceous composition or to test mutants in order to examine phosphoglycerate kinase activity at the molecular level.

Site-specific mutagenesis is a technique useful in the preparation of individual peptides, or biologically functional equivalent proteins, polypeptides or peptides, through specific mutagenesis of the underlying nucleic acid. The technique further provides a ready ability to prepare and test sequence variants, incorporating one or more of the foregoing considerations, by introducing one or more nucleotide sequence changes into the DNA. Site-specific mutagenesis allows the production of mutants through the use of specific oligonucleotide sequences which encode the nucleic acid sequence of the desired mutation, as well as a sufficient number of adjacent nucleotides, to provide a primer sequence of sufficient size and sequence complexity to form a stable duplex on both sides of the deletion junction being traversed. Typically, a primer of about 17 to 25 nucleotides in length is preferred, with about 5 to 10 residues on both sides of the junction of the sequence being altered.

In general, the technique of site-specific mutagenesis is well known in the art. As will be appreciated, the technique typically employs a bacteriophage vector that exists in both a single stranded and double stranded form. Typical vectors useful in site-directed mutagenesis include vectors such as the M13 phage. These phage vectors are commercially available and their use is generally well known to those skilled in the art. Double stranded plasmids are also routinely employed in site directed mutagenesis, which eliminates the step of transferring the gene of interest from a phage to a plasmid.

In general, site-directed mutagenesis is performed by first obtaining a single-stranded vector, or melting of two strands of a double stranded vector which includes within its sequence a nucleic acid sequence encoding the desired proteinaceous molecule. An oligonucleotide primer bearing the desired mutated sequence is synthetically prepared. This primer is then annealed with the single-stranded DNA preparation, and subjected to DNA polymerizing enzymes such as E. coli polymerase I Klenow fragment, in order to complete the synthesis of the mutation-bearing strand. Thus, a heteroduplex is formed wherein one strand encodes the original non-mutated sequence and the second strand bears the desired mutation. This heteroduplex vector is then used to transform appropriate cells, such as E. coli cells, and clones are selected that include recombinant vectors bearing the mutated sequence arrangement.

The preparation of sequence variants of the selected gene using site-directed mutagenesis is provided as a means of producing potentially useful species and is not meant to be limiting, as there are other ways in which sequence variants of genes may be obtained. For example, recombinant vectors encoding the desired gene may be treated with mutagenic agents, such as hydroxylamine, to obtain sequence variants.

As modifications and changes may be made in the structure of the phosphoglycerate kinase genes, nucleic acids (e.g., nucleic acid segments) and proteinaceous molecules of the present invention, and still obtain molecules having like or otherwise desirable characteristics, such biologically functional equivalents are also encompassed within the present invention.

For example, certain amino acids may be substituted for other amino acids in a proteinaceous structure without appreciable loss of interactive binding capacity with structures such as, for example, antigen-binding regions of antibodies, binding sites on substrate molecules or receptors, or such like. Since it is the interactive capacity and nature of a proteinaceous molecule that defines that proteinaceous molecule's biological functional activity, certain amino acid sequence substitutions can be made in a proteinaceous molecule sequence (or, of course, its underlying nucleic acid coding sequence) and nevertheless obtain a proteinaceous molecule with like (agonistic) properties. It is thus contemplated that various changes may be made in the sequence of phosphoglycerate kinase proteins, polypeptides or peptides, or the underlying nucleic acids, without appreciable loss of their biological utility or activity.

Equally, the same considerations may be employed to create a protein, polypeptide or peptide with countervailing, e.g., antagonistic properties. This is relevant to the present invention in which phosphoglycerate kinase mutants or analogues may be generated. For example, a phosphoglycerate kinase mutant may be generated and tested for its ability to inhibit or reduce uPAR activity or expression or to identify those residues important for its activity. Phosphoglycerate kinase mutants may also be synthesized to reflect a phosphoglycerate kinase mutant that occurs in the human population and that is linked to inflammatory diseases, cancer, wound healing and tissue scarring. Such mutant proteinaceous molecules are particularly contemplated for use in generating mutant-specific antibodies and such mutant DNA segments may be used as mutant-specific probes and primers.

While discussion has focused on functionally equivalent polypeptides arising from amino acid changes, it will be appreciated that these changes may be effected by alteration of the encoding DNA; taking into consideration also that the genetic code is degenerate and that two or more codons may code for the same amino acid. A table of amino acids and their codons is presented herein above for use in such embodiments, as well as for other uses, such as in the design of probes and primers and the like.

In terms of functional equivalents, it is well understood by the skilled artisan that, inherent in the definition of a “biologically functional equivalent” protein, polypeptide, peptide, gene or nucleic acid, is the concept that there is a limit to the number of changes that may be made within a defined portion of the molecule and still result in a molecule with an acceptable level of equivalent biological activity. Biologically functional equivalent peptides are thus defined herein as those peptides in which certain, not most or all, of the amino acids may be substituted.

In particular, where shorter length peptides are concerned, it is contemplated that fewer amino acids changes should be made within the given peptide. Longer domains may have an intermediate number of changes. The full length protein will have the most tolerance for a larger number of changes. Of course, a plurality of distinct proteins/polypeptide/peptides with different substitutions may easily be made and used in accordance with the invention.

It is also well understood that where certain residues are shown to be particularly important to the biological or structural properties of a protein, polypeptide or peptide, e.g., residues in binding regions or active sites, such residues may not generally be exchanged. In this manner, functional equivalents are defined herein as those peptides which maintain a substantial amount of their native biological activity.

Amino acid substitutions are generally based on the relative similarity of the amino acid side-chain substituents, for example, their hydrophobicity, hydrophilicity, charge, size, and the like. An analysis of the size, shape and type of the amino acid side-chain substituents reveals that arginine, lysine and histidine are all positively charged residues; that alanine, glycine and serine are all a similar size; and that phenylalanine, tryptophan and tyrosine all have a generally similar shape. Therefore, based upon these considerations, arginine, lysine and histidine; alanine, glycine and serine; and phenylalanine, tryptophan and tyrosine; are defined herein as biologically functional equivalents.

To effect more quantitative changes, the hydropathic index of amino acids may be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a proteinaceous molecule is generally understood in the art (Kyte and Doolittle, 1982). It is known that certain amino acids may be substituted for other amino acids having a similar hydropathic index or score and still retain a similar biological activity. In making changes based upon the hydropathic index, the substitution of amino acids whose hydropathic indices are within ±2 is preferred, those which are within ±1 are particularly preferred, and those within ±0.5 are even more particularly preferred.

It is also understood in the art that the substitution of like amino acids can be made effectively on the basis of hydrophilicity, particularly where the biological functional equivalent protein, polypeptide or peptide thereby created is intended for use in immunological embodiments, as in certain embodiments of the present invention. U.S. Pat. No. 4,554,101, states that the greatest local average hydrophilicity of a proteinaceous molecule, as governed by the hydrophilicity of its adjacent amino acids, correlates with its immunogenicity and antigenicity, i.e., with a biological property of the proteinaceous molecule.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

In making changes based upon similar hydrophilicity values, the substitution of amino acids whose hydrophilicity values are within ±2 is preferred, those which are within ±1 are particularly preferred, and those within ±0.5 are even more particularly preferred.

In addition to the phosphoglycerate kinase compounds described herein, it is contemplated that other sterically similar compounds may be formulated to mimic the key portions of the peptide structure. Such compounds, which may be termed peptidomimetics, may be used in the same manner as the peptides of the invention and hence are also functional equivalents.

Certain mimetics that mimic elements of proteinaceous molecule's secondary structure are described in Johnson et al. (1993). The underlying rationale behind the use of peptide mimetics is that the peptide backbone of proteinaceous molecules exists chiefly to orientate amino acid side chains in such a way as to facilitate molecular interactions, such as those of antibody and antigen. A peptide mimetic is thus designed to permit molecular interactions similar to the natural molecule.

Some successful applications of the peptide mimetic concept have focused on mimetics of β-turns within proteinaceous molecules, which are known to be highly antigenic. Likely β-turn structure within a polypeptide can be predicted by computer-based algorithms, as discussed herein. Once the component amino acids of the turn are determined, mimetics can be constructed to achieve a similar spatial orientation of the essential elements of the amino acid side chains.

The generation of further structural equivalents or mimetics may be achieved by the techniques of modeling and chemical design known to those of skill in the art. The art of receptor modeling is now well known, and by such methods a chemical that binds phosphoglycerate kinase can be designed and then synthesized. It will be understood that all such sterically designed constructs fall within the scope of the present invention.

In addition to the 20 “standard” amino acids provided through the genetic code, modified or unusual amino acids are also contemplated for use in the present invention. A table of exemplary, but not limiting, modified or unusual amino acids is provided herein (see Table 1).

TABLE 1
Modified and Unusual Amino Acids

| Abbr. | Amino Acid |
|---|---|
| Aad | 2-Aminoadipic acid |
| Baad | 3-Aminoadipic acid |
| Bala | Beta-alanine, beta-Amino-propionic acid |
| Abu | 2-Aminobutyric acid |
| 4Abu | 4-Aminobutyric acid, piperidinic acid |
| Acp | 6-Aminocaproic acid |
| Ahe | 2-Aminoheptanoic acid |
| Aib | 2-Aminoisobutyric acid |
| Baib | 3-Aminoisobutyric acid |
| Apm | 2-Aminopimelic acid |
| Dbu | 2,4-Diaminobutyric acid |
| Des | Desmosine |
| Dpm | 2,2′-Diaminopimelic acid |
| Dpr | 2,3-Diaminopropionic acid |
| EtGly | N-Ethylglycine |
| EtAsn | N-Ethylasparagine |
| Hyl | Hydroxylysine |
| aHyl | Allo-Hydroxylysine |
| 3Hyp | 3-Hydroxyproline |
| 4Hyp | 4-Hydroxyproline |
| Ide | Isodesmosine |
| aIle | Allo-Isoleucine |
| MeGly | N-Methylglycine, sarcosine |
| MeIle | N-Methylisoleucine |
| MeLys | 6-N-Methyllysine |
| MeVal | N-Methylvaline |
| Nva | Norvaline |
| Nle | Norleucine |
| Orn | Ornithine |

IV. Nucleic Acids Encoding Phosphoglycerate Kinase Peptides, Polypeptides, Proteins or Mutants or Mimetics Thereof

Important aspects of the present invention concern isolated DNA segments encoding wild-type, polymorphic or mutant phosphoglycerate kinase proteins, polypeptides or peptides, comprising the sequence of SEQ ID NO:1, and biologically functional equivalents thereof.

The present invention concerns DNA segments, isolatable from mammalian cells, such as mouse or human cells, that are free from total genomic DNA and that are capable of expressing a protein, polypeptide or peptide that has phosphoglycerate kinase activity. As used herein, the term “DNA segment” refers to a DNA molecule that has been isolated free of total genomic DNA of a particular species. Therefore, a DNA segment encoding phosphoglycerate kinase refers to a DNA segment that contains wild-type, polymorphic or mutant phosphoglycerate kinase coding sequences yet is isolated away from, or purified free from, total mammalian genomic DNA. Included within the term “DNA segment”, are DNA segments and smaller fragments of such segments, and also recombinant vectors, including, for example, plasmids, cosmids, phage, viruses, and the like.

Similarly, a DNA segment comprising an isolated or purified phosphoglycerate kinase gene refers to a DNA segment including phosphoglycerate kinase protein, polypeptide or peptide coding sequences and, in certain aspects, regulatory sequences, isolated substantially away from other naturally occurring genes or protein encoding sequences. In this respect, the term “gene” is used for simplicity to refer to a functional protein, polypeptide or peptide encoding unit. As will be understood by those in the art, this functional term includes both genomic sequences, cDNA sequences and engineered segments that express, or may be adapted to express, proteins, polypeptides, domains, peptides, fusion proteins and mutants of phosphoglycerate kinase encoded sequences.

The present invention contemplates the use of a nucleic acid(s) encoding a phosphoglycerate kinase peptide, polypetide, or protein. A “nucleic acid” as used herein includes single-stranded and double-stranded molecules, as well as DNA, RNA, chemically modified nucleic acids and nucleic acid analogs. It is contemplated that a nucleic acid within the scope of the present invention may be of about 20 to about 90, of about 100 to about 200, of about 210 to about 300, of about 310 to about 350, of about 360, to about 400, of about 410 to about 450, of about 460 to about 500, of about 510 to about 550, of about 560 to about 600, of about 610 to about 650, of about 660 to about 700, of about 710 to about 750, of about 760 to about 800, of about 810 to about 850, of about 860 to about 900, about 1000, about 1100, about 1200, about 1300, about 1400, about 1500, about 1750, about 2000, about 2250, about 2500 or greater nucleotide residues in length. Those of skill will recognize that in cases where the nucleic acid region encodes a phosphoglycerate kinase peptide, polypeptide or protein or mutant or mimetic thereof, the nucleic acid region can be quite long, depending upon the number of amino acids in the PGK molecule.

“Isolated substantially away from other coding sequences” means that the gene of interest, in this case the phosphoglycerate kinase gene, forms the significant part of the coding region of the DNA segment, and that the DNA segment does not contain large portions of naturally-occurring coding DNA, such as large chromosomal fragments or other functional genes or cDNA coding regions. Of course, this refers to the DNA segment as originally isolated, and does not exclude genes or coding regions later added to the segment by the hand of man.

In particular embodiments, the invention concerns isolated DNA segments that encode a phosphoglycerate kinase protein, polypeptide or peptide that includes within its amino acid sequence a contiguous amino acid sequence in accordance with, or essentially as set forth in, SEQ ID NO:2.

The term “a sequence essentially as set forth in SEQ ID NO:2” means that the sequence substantially corresponds to a portion of SEQ ID NO:2 and has relatively few amino acids that are not identical to, or a biologically functional equivalent of, the amino acids of SEQ ID NO:2.

The term “biologically functional equivalent” is well understood in the art and is further defined in detail herein. Accordingly, sequences that have about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%, and any range derivable therein, such as, for example, about 70% to about 80%, and more preferably about 81% and about 90%; or even more preferably, between about 91% and about 99%; of amino acids that are identical or functionally equivalent to the amino acids of SEQ ID NO:2 will be sequences that are “essentially as set forth in SEQ ID NO:2”, provided the biological activity of the protein is maintained.

In certain other embodiments, the invention concerns isolated DNA segments that include within their sequence a nucleic acid sequence essentially as set forth in SEQ ID NO:1. The term “essentially as set forth in SEQ ID NO:1” is used in the same sense as described above and means that the nucleic acid sequence substantially corresponds to a portion of SEQ ID NO:1, and has relatively few codons that are not identical, or functionally equivalent, to the codons of SEQ ID NO:1. Again, DNA segments that encode proteins, polypeptide or peptides exhibiting phosphoglycerate kinase activity will be most preferred.

The term “functionally equivalent codon” is used herein to refer to codons that encode the same amino acid, such as the six codons for arginine and serine, and also refers to codons that encode biologically equivalent amino acids. For optimization of expression of phosphoglycerate kinase in human cells, the codons are shown in Table 2 in preference of use from left to right. Thus, the most preferred codon for alanine is thus “GCC”, and the least is “GCG” (see Table 2 below). Codon usage for various organisms and organelles can be found on the internet at the Codon Usage Database website, allowing one of skill in the art to optimize codon usage for expression in various organisms using the disclosures herein. Thus, it is contemplated that codon usage may be optimized for other animals, as well as other organisms such as a prokaryote (e.g., an eubacteria, an archaea), an eukaryote (e.g., a protist, a plant, a fungi, an animal), a virus and the like, as well as organelles that contain nucleic acids, such as mitochondria or chloroplasts, based on the preferred codon usage as would be known to those of ordinary skill in the art.

TABLE 2
Preferred Human DNA Codons

| Amino Acids | | | Codons |
|---|---|---|---|
| Alanine | Ala | A | GCC GCT GCA GCG |
| Cysteine | Cys | C | TGC TGT |
| Aspartic acid | Asp | D | GAC GAT |
| Glutamic acid | Glu | E | GAG GAA |
| Phenylalanine | Phe | F | TTC TTT |
| Glycine | Gly | G | GGC GGG GGA GGT |
| Histidine | His | H | CAC CAT |
| Isoleucine | Ile | I | ATC ATT ATA |
| Lysine | Lys | K | AAG AAA |
| Leucine | Leu | L | CTG CTC TTG CTT CTA TTA |
| Methionine | Met | M | ATG |
| Asparagine | Asn | N | AAC AAT |
| Proline | Pro | P | CCC CCT CCA CCG |
| Glutamine | Gln | Q | CAG CAA |
| Arginine | Arg | R | CGC AGG CGG AGA CGA CGT |
| Serine | Ser | S | AGC TCC TCT AGT TCA TCG |
| Threonine | Thr | T | ACC ACA ACT ACG |
| Valine | Val | V | GTG GTC GTT GTA |
| Tryptophan | Trp | W | TGG |
| Tyrosine | Tyr | Y | TAC TAT |

It will also be understood that amino acid and nucleic acid sequences may include additional residues, such as additional N- or C-terminal amino acids or 5′ or 3′ sequences, and yet still be essentially as set forth in one of the sequences disclosed herein, so long as the sequence meets the criteria set forth above, including the maintenance of biological protein, polypeptide or peptide activity where an amino acid sequence expression is concerned. The addition of terminal sequences particularly applies to nucleic acid sequences that may, for example, include various non-coding sequences flanking either of the 5′ or 3′ portions of the coding region or may include various internal sequences, i.e., introns, which are known to occur within genes.

Excepting intronic or flanking regions, and allowing for the degeneracy of the genetic code, sequences that have about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%, and any range derivable therein, such as, for example, about 70% to about 80%, and more preferably about 81% and about 90%; or even more preferably, between about 91% and about 99%; of nucleotides that are identical to the nucleotides of SEQ ID NO:1 will be sequences that are “essentially as set forth in SEQ ID NO:1”.

One may also prepare fusion proteins, polypeptides and peptides, e.g., where the phosphoglycerate kinase proteinaceous material coding regions are aligned within the same expression unit with other proteins, polypeptides or peptides having desired functions, such as for purification or immunodetection purposes (e.g., proteinaceous compositions that may be purified by affinity chromatography and enzyme label coding regions, respectively).

Encompassed by the invention are DNA segments encoding relatively small peptides, such as, for example, peptides of from about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 35, about 40, about 45, to about 50 amino acids in length, and more preferably, of from about 15 to about 30 amino acids in length; as set forth in SEQ ID NO:2 and also larger polypeptides up to and including proteins corresponding to the full-length sequences set forth in SEQ ID NO:2, and any range derivable therein and any integer derivable therein such a range.

In addition to the “standard” DNA and RNA nucleotide bases, modified bases are also contemplated for use in particular applications of the present invention. A table of exemplary, but not limiting, modified bases is provided herein.

Where incorporation into an expression vector is desired, the nucleic acid encoding a phosphoglycerate kinase peptide, polypeptide or protein may also comprise a natural intron or an intron derived from another gene. It is contemplated in the present invention, that virtually any type of vector may be employed in any known or later discovered method to deliver nucleic acids encoding a phosphoglycerate kinase peptide, polypeptide or protein; constructs thereof including mutants and mimetic thereof. Such vectors may be viral or non-viral vectors as described herein, and as known to those skilled in the art. An expression construct comprising a nucleic acid encoding a phosphoglycerate kinase peptide, polypeptide, or protein or mutant or mimetic thereof may comprise a virus or engineered construct derived from a viral genome.

The ability of certain viruses to enter cells via receptor-mediated endocytosis and to integrate into the host cell genome and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986). The first viruses used as vectors were DNA viruses including the papovaviruses (simian virus 40, bovine papilloma virus, and polyoma) (Ridgeway, 1988; Baichwal and Sugden, 1986) and adenoviruses (Ridgeway, 1988; Baichwal and Sugden, 1986) and adeno-associated viruses. Retroviruses also are attractive gene transfer vehicles (Nicolas and Rubenstein, 1988; Temin, 1986) as are vaccina virus (Ridgeway, 1988) and adeno-associated virus (Ridgeway, 1988). Such vectors may be used to (i) transform cell lines in vitro for the purpose of expressing proteins of interest or (ii) to transform cells in vitro or in vivo to provide therapeutic polypeptides for gene therapy such as PGK peptides, polypeptides, proteins or mutants or mimetics thereof.

V. Rational Drug Design

In one aspect, any compound may be designed by rational drug design to function as a phosphoglycerate kinase molecule. The goal of rational drug design is to produce structural analogs of biologically active compounds. By creating such analogs, it is possible to fashion drugs which are more active or stable than the natural molecules, which have different susceptibility to alteration or which may affect the function of various other molecules. In one approach, one would generate a three-dimensional structure for the phosphoglycerate kinase protein of the invention or a fragment thereof. This could be accomplished by X-ray crystallography, computer modeling or by a combination of both approaches. An alternative approach, involves the random replacement of functional groups throughout the phosphoglycerate kinase protein, polypeptides or peptides, and the resulting affect on function determined.

It also is possible to isolate a phosphoglycerate kinase protein, polypeptide or peptide specific antibody, selected by a functional assay, and then solve its crystal structure. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

Thus, one may design drugs which have enhanced and improved, or reduced, biological activity, relative to a starting phosphoglycerate kinase proteinaceous sequences. By virtue of the ability to recombinatly produce sufficient amounts of the phosphoglycerate kinase proteins, polypeptides or peptides, crystallographic studies may be preformed to determine the most likely sites for mutagenesis and chemical mimicry. In addition, knowledge of the chemical characteristics of these compounds permits computer employed predictions of structure-function relationships. Computer models of various polypeptide and peptide structures are also available in the literature or computer databases. In a non-limiting example, the Entrez database may be used by one of ordinary skill in the art to identify target sequences and regions for mutagenesis.

VI. Screening for Modulators of uPAR Activity or Expression

The present invention comprises methods for identifying modulators of uPAR activity or expression. uPAR may be used as a target in screening for phopshoglycerate kinase compounds that inhibit, decrease, down-regulate or reduce its expression or activity in cells such as an inflammatory or cancer cell. These assays may focus on particular classes of compounds selected with an eye towards structural attributes that are believed to make them more likely to modulate the function of uPAR. In some instances, libraries may be randomly screened for candidate phopshoglycerate kinase substances. By function, it is meant that one may assay for inhibition of activity or expression of uPAR in inflammatory or neoplastic cells.

To identify a uPAR modulator, one generally will determine uPAR modulation in the presence and absence of the candidate substance, wherein a modulator is defined as a substance that has the ability to reduce, inhibit, or decrease, uPAR activity or expression. For example, a method may generally comprise:
a) providing a uPAR in a cell or a cell-free assay mixture;
b) contacting the uPAR with a candidate substance; and
c) measuring uPAR activity or expression,
   wherein a decrease in the uPAR activity or expression in the presence of the candidate substance as compared to the uPAR activity or expression in a cell or cell-free assay mixture not exposed to the candidate modulator indicates that the candidate substance has the ability to down-regulate or inhibit the expression or activity of uPAR.

Assays may be conducted in cell free systems, in isolated cells, or in organisms including transgenic animals. It will, of course, be understood that all the screening methods of the present invention are useful in themselves notwithstanding the fact that effective candidates may not be found. These assays may be performed at a lab bench by a human operator, via mechanized high through-put screening, or any other manner. The candidate substance(s) tested may be an individual candidate or one or more of a library of candidates and may be obtained from any source and in any manner known to those of skill in the art.

As used herein the term “candidate substance” or “candidate compound” refers to any PGK molecule that may potentially inhibit, reduce or decrease the activity or expression of uPAR. Any compound or molecule described in the methods and compositions herein may be an uPAR modulator. For example, the candidate substance may be PGK a peptide, polypeptide, protein, or mutant or mimetic thereof, or fragment thereof, a small molecule, even a nucleic acid molecule.

VII. Pharmaceutical Compositions and Delivery of Phosphoglycerate Kinase Peptide, Polypeptide, Protein or Mutant or Mimetic Thereof

In an embodiment of the present invention, a method for treating an inflammatory and/or neoplastic disease by the delivery of a phosphoglycerate kinase molecule to a subject is contemplated. A method of promoting wound healing and preventing scarring is also contemplated in the present invention. Inflammatory diseases that are most likely to be treated in the present invention include but are not limited to those of the lung, thyroid, larynx, bladder, colon, esophagus, gastrointestine, gum, nasopharynx, or skin. In further embodiments, neoplastic diseases that are contemplated for treatment include but are not limited to cancers of the lung, breast, head and neck, bladder, bone, bone marrow, brain, colon, esophagus, gastrointestine, gum, kidney, liver, nasopharynx, ovary, prostate, skin, stomach, testis, tongue, or uterus.

A. Delivery of Nucleic Acids Encoding PGK Peptides, Polypeptides, Proteins, Mutants and/or Mimetics

In some embodiments of the present invention, a method for treating an inflammatory and/or neoplastic disease by delivering a nucleic acid encoding a phosphoglycerate kinase peptide, polypeptide, protein, mutant or mimetic thereof, to a subject is contemplated. Such nucleic acids may be comprised in any form of viral, plasmid, or other vector described herein or known to those of skill. Typically, appropriate control sequences will surround the coding regions of the nucleic acids. Virtually any method by which nucleic acids can be introduced into a cell, or an organism may be employed with the current invention, as described herein or as would be known to one of ordinary skill in the art. Such methods include, but are not limited to direct delivery of DNA by: injection (U.S. Pat. Nos. 5,994,624, 5,981,274, 5,945,100, 5,780,448, 5,736,524, 5,702,932, 5,656,610, 5,589,466 and 5,580,859, each incorporated herein by reference); microinjection (Harland and Weintraub, 1985; U.S. Pat. No. 5,789,215, incorporated herein by reference); electroporation (U.S. Pat. No. 5,384,253, incorporated herein by reference; Tur-Kaspa et al., 1986; Potter et al., 1984); calcium phosphate precipitation (Graham and Van Der Eb, 1973; Chen and Okayama, 1987; Rippe et al., 1990); using DEAE-dextran followed by polyethylene glycol (Gopal, 1985); direct sonic loading (Fechheimer et al., 1987); liposome mediated transfection (Nicolau and Sene, 1982; Fraley et al., 1979; Nicolau et al., 1987; Wong et al., 1980; Kaneda et al., 1989; Kato et al., 1991) receptor-mediated transfection (Wu and Wu, 1987; Wu and Wu, 1988); microprojectile bombardment (PCT Applications WO 94/09699 and 95/06128; U.S. Pat. Nos. 5,610,042; 5,322,783 5,563,055, 5,550,318, 5,538,877 and 5,538,880, and each incorporated herein by reference); agitation with silicon carbide fibers (Kaeppler et al., 1990; U.S. Pat. Nos. 5,302,523 and 5,464,765, each incorporated herein by reference); Agrobacterium-mediated transformation (U.S. Pat. Nos. 5,591,616 and 5,563,055, each incorporated herein by reference); PEG-mediated transformation of protoplasts (Omirulleh et al., 1993; U.S. Pat. Nos. 4,684,611 and 4,952,500, each incorporated herein by reference); desiccation/inhibition-mediated DNA uptake (Potrykus et al., 1985); or any combination of such methods.

B. Injectable Compositions and Formulations

Phosphoglycerate kinase peptides, polypeptides, proteins, or a mutant or a mimetic as in the present invention may be administered by any method known to those of skill in the art. They may be preferably administered intravenously, intralesionally, precutaneously, subcutaneously or inhalation to a subject for the inhibit, or reduce uPAR activity and/or expression. However, the pharmaceutical compositions disclosed herein may alternatively be administered parenterally, intrapleurally, intradermally, intramuscularly, transdermally or even intraperitoneally as described in U.S. Pat. Nos. 5,543,158; 5,641,515 and 5,399,363.

Injection of phosphoglycerate kinase and its related compounds may be administered by syringe or any other method used for injection of a solution, as long as the molecules can pass through the particular gauge of needle required for injection. A novel needleless injection system has recently been described (U.S. Pat. No. 5,846,233) having a nozzle defining an ampule chamber for holding the solution and an energy device for pushing the solution out of the nozzle to the site of delivery. A syringe system has also been described for use in gene therapy that permits multiple injections of predetermined quantities of a solution precisely at any depth (U.S. Pat. No. 5,846,225).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, intratumoral and intraperitoneal administration. In this connection, sterile aqueous media that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The compositions disclosed herein may be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug release capsules and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically-acceptable” or “pharmacologically-acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared.

C. Dosage and Schedules of Administration

The dosage of the active compound and dosage schedule may be varied on a subject by subject basis, taking into account, for example, factors such as the weight and age of the subject, the type of disease being treated, the severity of the disease condition, previous or concurrent therapeutic interventions, the manner of administration and the like, which can be readily determined by one of ordinary skill in the art.

Administration is in any manner compatible with the dosage formulation, and in such amount as will be therapeutically effective. The quantity to be administered depends on the subject to be treated, including, e.g., the capacity of the individual's immune system to synthesize antibodies, and the degree of protection desired. The dosage of the will depend on the route of administration and will vary according to the size of the host. Precise amounts of an active ingredient required to be administered depend on the judgment of the practitioner.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein However, a suitable dosage range may be, for example, of the order of several hundred micrograms active ingredient per vaccination. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per vaccination, and any range derivable therein. In non-limiting examples of a derivable range from the numbers listed herein, a range of about 5 mg/kg/body weight to about 100 mg/kg/body weight, about 5 microgram/kgjbody weight to about 500 milligram/kg/body weight, etc., can be administered, based on the numbers described above. A suitable regime for initial administration and booster administrations (e.g., inoculations) are also variable, but are typified by an initial administration followed by subsequent inoculation(s) or other administration(s).

In many instances, it will be desirable to have multiple administrations, usually not exceeding six administrations, more usually not exceeding four administrations and preferably one or more, usually at least about three administrations. Normally from two to twelve week intervals, more usually from three to five week intervals may be applicable.

### VIII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

Materials and Methods

Material. Culture media, penicillin, streptomycin, fetal calf serum (FCS) were purchased from Gibco BRL laboratories (Grand Island, N.Y.); tissue culture plastics were from Becton Dickinson Labware (Lincoln Park, N.J.). Herbimycin from Calbiochem (La Jolla, Calif.), bovine serum albumin (BSA), ovalbumin, Tris-base, aprotinin, dithiothreitol (DTT), phenylmethylsulfonyl fluoride (PMSF) and ammonium persulfate (APS) were from Sigma Chemical Company (St. Louis, Mo.). Acrylamide, bisacrylamide and nitrocellulose were from BioRad laboratories (Richmond, Calif.). Anti-uPAR antibody was obtained from American Diagnostics (Greenwich, Conn.). XAR X-ray film was purchased from Eastman Kodak (Rochester, N.Y.).

Cell Cultures. Human bronchial epithelial cells (Beas2B) and squamous cell carcinoma (H157) were maintained in LHC-9, and RPMI 1640 medium containing 10% heat-inactivated FCS, 1% glutamine and 1% antibiotics as previously described (Shetty et al., 1995).

Purification of the uPAR mRNABp from Beas2B Cells. Beas2B cells grown to confluence were serum starved overnight in RPMI media. The cells were collected by treating with trypsin/EDTA, washed using ice-cold HBSS. The cell were lysed in lysis buffer on ice for 1 h, after which the lysate was centrifuged at 15,000 rpm for 15 min. The clear supernatant was used as a cytosolic extract. This cytosolic extract was further fractionated to 0-50%, 50-80% and 80-100% by ammonium sulfate fractionation. These fractions were later subjected to uPAR mRNA binding activity by gel mobility and Northwestern assays. Protein extracts from 80-100% ammonium sulfate fractionation was loaded onto a heparin affigel column and bound proteins were eluted with 100 ml 0-1000 mM linear gradient of NaCl. Fractions were desalted by dialysis and subjected to uPAR mRNA binding analysis as described above. Active fractions were equilibrated with 1.7M ammonium sulfate and loaded onto a phenyl sepharose column. The bound proteins were eluted from the phenyl sepharose column using ammonium sulfate reverse gradient and used for uPAR mRNA binding after dialysis. The positive fractions were concentrated using polyethylene glycol and loaded onto Mono-Q column connected to a FPLC system. Bound proteins were eluted by NaCl gradient, after which positive fractions were pooled and loaded on to Mono-Q columns for a second time, then collected in an identical fashion.

Peptide Sequencing. Active fractions containing RNA-binding activity were separated on SDS-PAGE, then transferred to a PVDF membrane. Tryptic peptides were obtained from protein blotted onto Pro Blott membranes (Applied Biosystems). Peptides were purified by reverse-phase HPLC and sequenced. The N-terminal sequence was determined and this sequence was used for peptide Blasts searches. The longest amino acid peptide resulted in 83% homology to PGK.

PCR™ Cloning. PGK cDNA was generated by PCR™ using poly (a) RNA from Beas2B cells and cloned to pcDNA3.1D/V5-His-Topo vector. Sequence and orientations were confirmed by nucleotide sequencing. Alternatively, the inventors also cloned this cDNA to a pET-DEST42 plasmid vector and transfected into BL21 gold. The protein expression was induced by IPTG and native PGPK fusion protein was purified by Ni-NTA-His column.

Transfection of H157 Cells. The H157 cells were transfected with or without PGK cDNA in pcDNA3.1DNV5-His-TOPO or empty vector pcDNA-Dest40 by lipofection as described earlier. The stable cell lines were generated by antibiotic selection and the cells were cultured in large amount and expression was confirmed by Western blotting using anti-V5 antibody. The recombinant fusion proteins were affinity purified by passing the lysate to Nickel column and subjected to uPAR mRNA binding studies by gel mobility shift and Northwestern assay as described below.

Plasmid Construction. Plasmid uPAR/pBluescript was obtained from the ATCC. The human uPAR mRNA template containing a complete sequence of uPAR cDNA (nucleotides −16 to 1144) from uPAR pbluescript was subcloned to Hind III and Xba I sites of pRC/CMV (Invitrogen) and the sequences of the clones were confirmed by sequencing.

In vitro Transcription. Linearized plasmids containing the human uPAR mRNA transcriptional template of complete uPAR cDNA were transcribed in vitro with T7 or Sp6 polymerase (Ambion). The uPAR mRNA transcripts were synthesized according to the supplier's protocol except that 50 μCi of [³²-P]UTP was substituted for unlabeled UTP in the reaction mixture. Passage through a Sephadex G-25 column removed unincorporated radioactivity. The specific activities of the product were 4.9×10⁸.

Gel Mobility Shift Assay. Eluate purified from Nickel column of lysates of H157 cells, transfected with PGK or vector cDNA was incubated with 2×10⁴ cpm of ³²P-labeled transcript in a mixture containing 15 mM KCl, 5 mM MgCl₂, 0.25 mM dithiothreitol, 12 mM Hepes (pH 7.9), 10% glycerol and Escherichia coli tRNA (200 ng/μl) in a total volume of 20 μl at 30° C. for 30 min. The reaction mixture were treated with 50 U of RNase T, or A and incubated at 37° C. for 30 min. To avoid nonspecific binding, 5 mg of heparin per ml was added, and the mixture was incubated at room temperature for an additional 10 min. Samples were separated by electrophoresis on a 5% native polyacrylamide gel with 0.25× tris-borate-EDTA running buffer. The gels were dried and developed by autoradiography at −70° C.

Northwestern Assay. Northwestern assay confirmed the molecular weight and uPAR mRNA-protein interaction. Protein eluate from Nickel column was separated on 8% SDS-PAGE, blotted to nitocellulose membrane. The membrane was blocked with gel shift buffer containing 1% BSA and 20 μg ribosomal RNA for 1 h. The membrane was replaced with fresh buffer containing ³²P-labeled uPAR mRNA (200000 cpm/ml) and incubated for an additional 1 h at RT. The membrane was later washed thrice with 50 ml of gelshift buffer for 10 min each, air dried and exposed to X-ray film. The membrane was later stripped and developed by Western blot using a β-actin monoclonal antibody as described above for equal loading.

Total Cellular Membrane Extraction and Western Blotting. H157 cells as well as stable cell lines of PGK or vector transfected cells grown to confluence were serum starved overnight with RPMI-glutamine media containing 0.5% BSA. The cells were washed with PBS. Receptor bound uPA was removed by glycine-HCl treatment as described earlier (Shetty et al., 1995a; Shetty et al., 1995b). SDS gel electrophoresis and Western blotting were used to measure functional uPAR at the cell surface. Membrane proteins isolated as described earlier (Shetty et al., 1995a; Shetty et al., 1995b) from the cells were separated by SDS-PAGE and transferred to a nitrocellulose membrane. The membrane was blocked with 1% BSA in wash buffer for 1 h at room temperature followed by overnight hybridization with uPAR monoclonal antibody in the same buffer at 4° C., washed and uPAR proteins were detected by enhanced chemiluminescence (ECL).

Random Priming of uPAR cDNA. The full length template of uPAR was released with Hind III or Xba I, purified on 1% agarose gels and labeled with ³²P-dCTP using a rediPrime labeling kit (Amersham, Arlington Heights, Ill.). Passage through a Sephadex G-25 column removed unincorporated radioactivity. The specific activity of the product was 6×10 cpM/μg.

Northern Blotting of uPAR mRNA. A Northern blotting assay was used to assess the level of uPAR mRNA. H157 cells grown to confluence was serum-starved overnight in RPMI-BSA media. Total RNA was isolated using TRI reagent. RNA (20 μg) was isolated on agarose/formaldehyde gels. After electrophoresis, the RNA was transferred to Hybond N⁺ according to the instructions of the manufacturer. Prehybridization and hybridization was done at 65° C. in NaCl (1M) /SDS (1%) and 100 μg/ml salmon sperm DNA. Hybridization was performed with a uPAR cDNA probe (1 ng/ml) labeled to approximately 6×10⁸ cpm/μg of DNA overnight. After hybridization, the filters were washed twice for 15 min at 65° C., with: 2×SSC, 1% SDS; 1×SSC, 1% SDS, and 0.1% SSC, 1% SDS, respectively. The membranes were next exposed to X-ray film at −70° C. overnight. The intensity of the bands was measured by densitometry and normalized against that of β-actin. The stability of uPAR mRNA was also measured in these cell lines by transcription chase studies as described earlier (Shetty et al., 1997).

DNA Synthesis. H157 and stable cell lines transfected with PGK cDNA in pcDNA3.1 or vector alone were grown to confluence in 24 well plates. The cells were serum starved overnight in SAEC basal or RPMI medium containing 0.5% BSA. ³H-thymidine was later added to the same media and incubated for an additional 6 h. The cells were washed with ice cold PBS three times and five washes with ice cold 5% TCA. The cells were lysed in 0.2N NaOH and the incorporated ³H-thymidine was then counted by scintillation counter.

Cell Migration/Invasion Assay. Cell migration assays were performed using modified Boyden Chambers with 6.5 mm diameter, 10 μm thickness, porous (8.0 μm) polycarbonate membrane separating two chambers (Transwell®, Costar, Cambridge Mass., USA). Cells were added at a final concentration of 10,000 cells to upper chamber in RPMI (10% FCS) media. The cells were incubated for 72 h at 37° C. At the end of the assay the cells from the upper and lower chamber were detached by trypsin/EDTA. The cells which migrated to the lower chamber and those remaining in the upper chamber were counted using a Coulter counter. The percentage of cells in the upper and lower chambers were calculated based on the total number of cells present in upper and lower chambers. The invasion/migration ratio (IMR) was calculated as a ratio of the percent cells in the lower chamber versus the upper chamber, as previously described (Shetty and Idell, 1998, 1999).

### Example 2

Purification of uPAR Coding Region mRNA Binding Protein

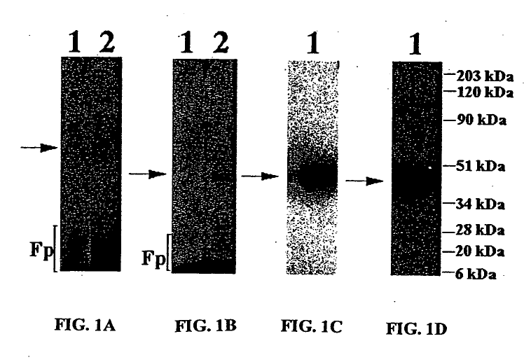

In order to purify the uPAR coding region mRNA binding protein from Beas2B cells, the inventors used conventional chromatography combined with gel mobility shift and Northwestern assay to track uPAR mRNA binding activity. An 80-100% ammonium sulfate fraction was passed through heparin sepharose column and the bound proteins were eluted and tested for uPAR CDR mRNA binding activity. The positive heparin resin fractions were then loaded onto a phenyl sepharose column and positive fractions from this column were next subjected to Mono-Q column chromatography using an FPLC system. The positive uPAR mRNA binding fractions were collected and subjected a second fractionation using the same column. Semipurified fractions were next subjected to a uPAR mRNA binding using gel mobility shift assay and RNA-protein complexes were excised from the gel and pooled after which the protein was extracted by electroelution. The eluted protein was later subjected to uPAR CDR mRNA binding by gel mobility shift, UV cross-linking and Northwestern assays (FIG. 1). As shown FIG. 1, this eluted protein formed a specific complex with the uPAR mRNA coding region.

To characterize the uPAR mRNA-binding protein, a protein purified from Beas2B cells was identified by microsequencing. The electroeluate was separated by SDS-PAGE and developed with silver staining, which showed a major species with an approximate molecular weight of 50 kDa. The mobility pattern was consistent with the uPAR CDR binding activity seen on Northwestern assay indicating this protein species interacts with uPAR mRNA. The N-terminal end of the purified protein was microsequenced, revealing a protein species of 50 kDa demonstrating 83% homology to phosphoglycerate kinase (PGK) (Michelson et al. 1983). PGK has been shown to possess a wide range of biological activities in addition to its well-characterized role in glycolysis. Particularly, PGK is a key glycolytic enzyme that catalyzes the reversible conversion of 1,3-diphosphoglycerate to 3-phosphoglycerate. This protein shares a common nucleotide binding (either ATP or NAD) binding domain in combination with unique catalytic domains.

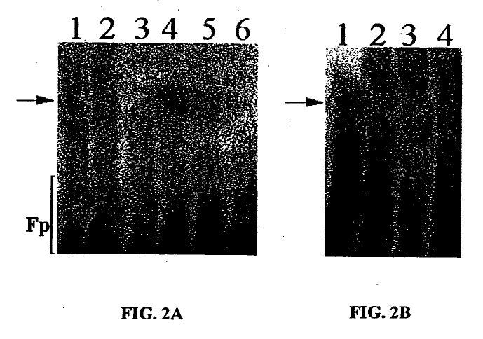

Primers corresponding to entire PGK coding region were designed for PCR™ cloning. A 1200-nt clone was amplified From Beas2B cells and this fragment was subjected to a directional cloning using the pENTER directional TOPO cloning kit (Invitrogen). The PCR™ product was sequenced to confirm orientation. This was later subjected to subcloning in a eukaryotic expression vector pcDNA3.1 DNV5-His-TOPO as well as prokarytotic expression pET-Dest 42 vector. The constructs were transfected to eukaryotic (H157) and prokaryotic (BL21) cells respectively. Stable cell lines were created by treating the transfected cells with G418 and these cells were cultured in large culture dishes. In BL21 cells, the native PGK protein was induced by IPTG and the inventors confirmed the expression of fusion protein by Western blotting using an anti-V5-monoclonal antibody (FIG. 2). Fusion proteins were purified by Ni-NTA-His column (Qiagen).

The native proteins were subjected to gel mobility shift and Northwestern assays to confirm uPAR mRNA binding activity. As shown FIG. 2, PGK expressed in both prokaryotic and eukaryotic cells showed uPAR mRNA binding activity by gel mobility and Northwestern assays.

### Example 3

Determination of PGK Interaction with uPAR

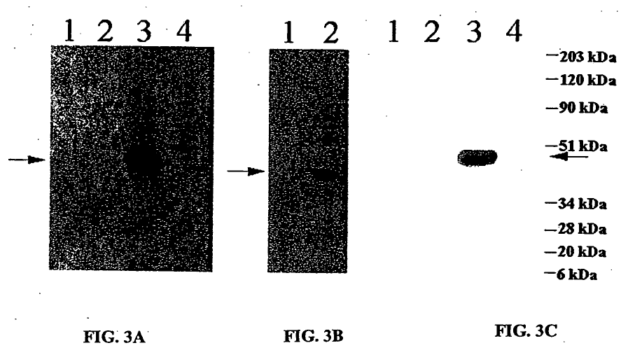

Since PGK specifically interacted with uPAR mRNA an analysis was next carried out to determine whether this interaction regulates cell surface uPAR expression. To confirm this hypothesis, cultured stable H157 cells were transfected with empty vector Dest40 (D40) or PGK cDNA in D40 (PGK) separately in culture dishes. H157 cells were also used without any transfection as controls. As shown in FIG. 3A, transfection of PGK cDNA reduced cell surface uPAR expression compared to vector transfected or control H157 cells. These data strongly suggest the possibility that PGK regulates expression of cell surface uPAR expression in H157 cells via regulation of the levels of cellular uPAR mRNA. To address this likelihood, the effect of PGK on uPAR mRNA expression in these cells was determined. As shown in FIG. 3B, H157 cells overexpressing PGK cDNA inhibited uPAR mRNA expression compared to control H157 cells or H157 cells transfected with empty vector. These data show that PGK mediated inhibition of cell surface uPAR expression is due to decreased uPAR mRNA expression.

### Example 4

PGK-mediated Inhibition of uPAR mRNA Expression

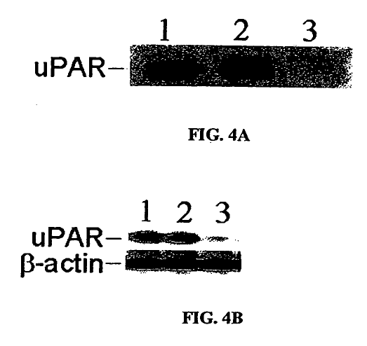

The inventors next confirmed whether PGK-mediated inhibition of uPAR mRNA expression in H157 cells is due to decreased mRNA synthesis or enhanced mRNA degradation. Therefore, H157 cell lines were cultured in T170 flasks and intact nuclei isolated; the lysates were then subjected to nuclear run-on assays. The results of nuclear run-on assays indicated that there is no significant change in the rate of uPAR mRNA transcription in PGK cDNA transfected cells (FIG. 4A). Since rate of uPAR mRNA synthesis was not altered, the inventors inferred that PGK probably regulates uPAR expression at the posttranscriptional level. To confirm this inference, confluent H157 cells were treated with DRB to inhibit ongoing transcription and analyzed uPAR mRNA expression by Northern blotting. As shown FIG. 4B, uPAR mRNA is quite stable in H157 cells with a half-life of 6-8 h. Conversely, uPAR mRNA was degraded much faster in PGK over-expressing cells.

### Example 5

Assessing the Effect of PGK Over-expression on the Rate of Cell Proliferation

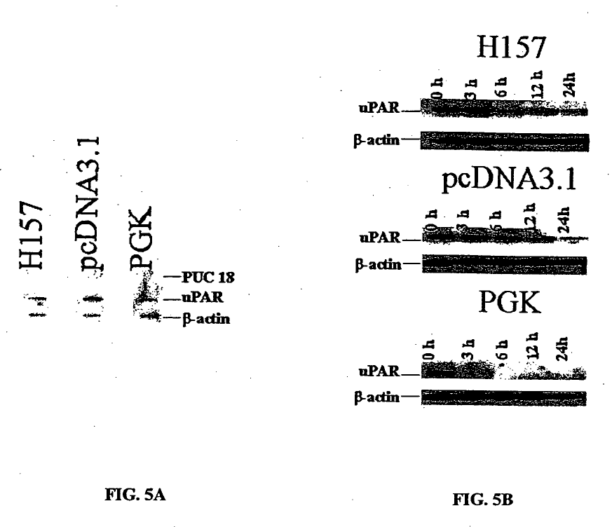

Since cellular proliferation of H157 cells is influenced by differences in uPAR expression, the effect PGK over-expression on the rate of proliferation of H157 cells was assessed by ³H-thymidine incorporation. ³H-thymidine uptake by subconfluent monolayers of cells transfected with vector alone or PGK was compared with control H157 cells. As shown in FIG. 5A, PGK over-expression inhibited ³H-thymidine uptake by at least 50% in H157 cell compared to vector transfected or control H157 cells. Having confirmed that PGK regulates proliferation of H157 cells, these cells were studied in invasion/migration assays, which is likewise influenced by cellular uPAR expression (Shetty and Idell, 1999). As shown in FIG. 5B, PGK over-expressing cells exhibited decreased migration by at least 40% compared to control cells used in the assays.

### Example 6

In vitro Screening of PGK for Inhibition of uPAR Activity

Those of skill can employ the teachings of this specification to test for the ability of any PGK peptide, polypeptide, protein, mutant or mimetic in the context of the inhibition of uPAR activity and, therefore, therapeutic utility in the context of the invention. In this regard, the PGK peptide, polypeptide, protein, mutant or mimetic may be one specifically described in this specification, known to those of skill in the art, or made according to the teachings of this specification and/or knowledge of those of skill.

In vitro assays may be used in accordance with the invention to determine inhibition of uPAR activity or expression by any such PGK peptide, polypeptide, protein, mutant or mimetic. Those of skill in the art will, in view of this specification, understand that any of a number of in vitro assays can be used in this regard, including, but not limited to the assays described herein. For example, genetically altered or engineered cell lines, for example, lung cancer cell lines, in which uPAR or the uPAR receptor is overexpressed may be contacted with an effective amount of a PGK peptide, polypeptide, protein, mutant or mimetic and examined for the inhibition of expression or activity of uPAR. The inhibition of expression of uPAR or uPAR receptor may be analyzed by western or northern blotting.

Thus, using the teachings of the specification, one of ordinary skill in the art will be able to test any PGK molecule or PGK-related molecule in vitro for its ability to inhibit uPAR receptor expression or uPAR activity or expression and thus establish its utility as a therapeutic agent.

### Example 7

In Vivo Testing of the Ability of PGK to Inhibit uPAR Activity

Using the teachings of the present invention as disclosed herein the ability of any PGK peptide, polypeptide, protein, mutant or mimetic to regulate the expression of uPAR may be assessed using in vivo models. Typically, such in vivo testing will result occur after a PGK peptide, polypeptide, protein, mutant or mimetic has been validated in in vitro tests.

Typically in such in vivo testing, one or more unit dose of a PKG peptide, polypeptide, protein, mutant or mimetic will be administered (e.g., injected into) to an animal, for example, mice, afflicted with an inflammatory disease such as psoriasis, or having an inflammation after an injury, or a neoplastic disease in which uPAR or its receptor is expressed. Those of skill in the art understand that there are a wide variety of animal models for these conditions, and will be able to determine appropriate animal models in order to test for the effect of a PGK peptide, polypeptide, protein, mutant or mimetic in treating a given condition or disease state. In some instances a scid mouse model, such as a cancer model may be utilized, including, but not limited to models for ovarian, breast, lung, or other cancers. In some cases, preclinical models of lung or pleural injury in the rabbit, mouse or in other species including the baboon or other primates may be utilized. Those of skill in the art will be able to determine and test appropriate dosages and timing of application regimes for these studies, as well as appropriate manners in which to deliver the PGK peptide, polypeptide, protein, mutant or mimetic. Animals will typically be monitored before, during, and after the treatment regime, to determine any effects of the treatment on the condition or disease state. In some cases, examination will involve visual examination of living animals. In other cases, animals will be sacrificed at relevant times and tissue from an afflicted area or organ will be analyzed visually and/or microscopically.

Similar experiments will be conducted using PKG-related molecules that inhibit activity and/or expression of uPAR or the expression of the uPAR receptor, and that may have utility as a therapeutic agent for treating an inflammatory disease and/or a neoplastic disease.

### Example 8

Clinical Trials

Those of ordinary skill in the art will also be able to conduct appropriate clinical testing of PGK peptide, polypeptide, protein, mutant or mimetic molecules in inhibiting uPAR activity or expression and treating the disease states and conditions discussed herein. For example, a PKG peptide, polypeptide, protein, mutant or mimetic may be employed in clinical trials to confirm its efficacy as clinical treatment for cancers overexpressing uPAR or uPAR receptor, inflammatory diseases, and/or inflammation resulting from injury. Such clinical treatment will take into consideration various parameters involved in conducting a clinical trail, including patient treatment and monitoring. Such a clinical trial will be conducted by the skilled practitioner following the appropriate guidelines for clinical trials and taking into consideration variables such as the age and body weight of the individuals, and grade or stage of the disease state or condition.

The PKG peptide, polypeptide, protein, mutant or mimetic may be administered in the pleural space, intratumorally or systemically. In some instances, the PKG peptide, polypeptide, protein, mutant or mimetic of the present invention may be administered in combination with other active agents relevant to the disease state or condition. Such an agent may be administered before, after, or at the same time as the PKG peptide, polypeptide, protein, mutant or mimetic taking into consideration the dosage and toxicity of the agent.

The efficacy of the therapeutic treatment of the PGK peptide, polypeptide, protein, mutant or mimetic of the present invention may be assessed using any method known to one of skill in the art. The efficacy of the compounds of the invention may be indicated by any reduction in the disease state or condition, as measured by relevant means known to those of skill in the art in view of the specific disease state or condition.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

References

The following references, to the extent that they provide exemplary procedural or other details supplementary to those set forth herein, are specifically incorporated herein by reference.
U.S. Pat. No. 4,554,101
U.S. Pat. No. 4,684,611
U.S. Pat. No. 4,952,500
U.S. Pat. No. 5,302,523
U.S. Pat. No. 5,322,783
U.S. Pat. No. 5,384,253
U.S. Pat. No. 5,399,363
U.S. Pat. No. 5,464,765
U.S. Pat. No. 5,466,468
U.S. Pat. No. 5,538,877
U.S. Pat. No. 5,538,880
U.S. Pat. No. 5,543,158
U.S. Pat. No. 5,550,318
U.S. Pat. No. 5,563,055
U.S. Pat. No. 5,580,859
U.S. Pat. No. 5,589,466
U.S. Pat. No. 5,591,616
U.S. Pat. No. 5,610,042
U.S. Pat. No. 5,641,515
U.S. Pat. No. 5,656,610
U.S. Pat. No. 5,702,932
U.S. Pat. No. 5,736,524
U.S. Pat. No. 5,780,448
U.S. Pat. No. 5,789,215
U.S. Pat. No. 5,846,225
U.S. Pat. No. 5,846,233
U.S. Pat. No. 5,945,100
U.S. Pat. No. 5,981,274
U.S. Pat. No. 5,994,624
Achbarou et al., Cancer Res., 54:2372-2377, 1994.
Baichwal and Sugden, In: Gene Transfer, Kucherlapati (Ed.), New York, Plenum Press, 117-148, 1986.
Berg et al., Life Sci. Adv. Mol. Biol., 12:85-91, 1993.
Bhat et al., Am. J. Physiol., 277: L301-L309, 1999.
Blasi et al., J. Cell Biol., 104:801-804, 1987.
Boeret al., Mol. Cell Biol., 7:3107-3112, 1987.
Boyd et al., Cancer Res., 48:3112-3116, 1988.
Chapman et al., Chest, 93:1256-1263, 1988.
Chapman, Cur. Opin. Cell Biol., 9:714-724, 1997.
Chen and Okayama, Mol. Cell Biol., 7(8):2745-2752, 1987.
Chu et al., Biochemistry, 32(18):4756-4760, 1993.
Clerch et al., FEBS Lett., 328(3):250-252, 1993.
Dano et al., Adv. Cancer Res., 44:139-266, 1985.
Dvorak et al., Cancer Met. Rev., 2:41-73, 1983.
Dvorak, New Engl. J. Med., 315:1650-1659, 1986.
Elzinga et al., Nucleic Acids Res., 21(23):5328-5331, 1993.
Fechheimer et al., Proc. Natl. Acad. Sci. USA, 84:8463-8467, 1987.
Fraley et al., Proc. Natl. Acad. Sci. USA, 76:3348-3352, 1979.
Gebara and McCarrey, Mol. Cell Biol., 12:1422-1431, 1992.
Ghiso et al., Eur. J. Biochem., 263:295-304, 1999.
Gopal, Mol. Cell. Biol., 5:1188-1190, 1985.
Graham and Van Der Eb, Virology, 52:456-467, 1973.
Harland and Weintraub, J. Cell Biol., 101:1094-1099, 1985.
Hentze, Trends Biochem. Sci., 19(3):101-103, 1994.
Hollas et al., Thromb. Haemost., 68:662-666, 1992.
Idell et al., Am. J. Physiol., 267:L693-L703, 1994.
Idell, In: Lung Biology in Health and Disease, Phan and (Eds.), Marcel Dekker, NY, 80:743-776, 1995.
Idell, New Horizons, 2:566-574, 1994.
Janicke et al., Sem. Thromb. Hemost., 17:303-312, 1991.
Johnson et al., In: Biotechnology And Pharmacy, Pezzuto et al. (Eds.), Chapman and Hall NY, 1993.
Jones et al., Cell, 42:559-572, 1985.
Kadonaga et al., Cell, 51:1079-1090, 1987.
Kaeppler et al., Plant Cell Reports, 9:415-418, 1990.
Kaneda et al., Science, 243:375-378, 1989.
Kato et al, J. Biol. Chem., 266:3361-3364, 1991.
Kramer and Erickson, Dev. Biol., 87:37-45, 1981.
Kumari etal., J. Biol. Chem., 271:14390-14397, 1996.
Kyte and Doolittle, J. Mol. Biol., 157(1):105-132, 1982.
Liotta et al., Cancer Invest., 9:543-551, 1991.
Lund et al., Biochem. J., 310:345-352, 1995.
Lund et al., J. Biol. Chem., 266:5177-5181, 1991.
Mazar et al., Angiogenesis, 3:15-32, 1999.
McCarrey and Thomas, Nature (Lond), 326:501-505, 1987.
McCarrey Gene, 61:291-298, 1987.
McCarrey et al., Dev. Biol., 154:160-168, 1992.
McCarrey et al., Genet. Anal. Tech. Appl., 9:117-123, 1992.
McCarrey et al., Dev. Genet., 19:321-332, 1996.
McCarrey, Bioscience, 44:20-27, 1994.
McCarrey, Nucleic Acids Res., 18:949-955, 1990.
Michelson et al., J. Biol. Chem., 260:6982-6992, 1985.
Michelson et al., Proc. Natl. Acad. Sci. USA, 80(2):472-6., 1983.
Mignatti and Rifkin, Physiol. Rev., 73:161-195, 1993.
Mitsubayashi et al., Urol. Res., 15:335-339, 1987.
Nagy and Rigby, J. Biol. Chem., 270(6):2755-2763, 1995.
Nakagawa et al., Proc. Natl. Acad. Sci. USA, 92(6):2051-5, 1995.
Nanbu et al., J. Biochem. (Tokyo), 114(3):432-437, 1993.
Nicolas and Rubenstein, In: Vectors: A survey of molecular cloning vectors and their uses, Rodriguez and Denhardt (Eds.), Stoneham: Butterworth, 493-513, 1988.
Nicolau and Sene, Biochim. Biophys. Acta, 721:185-190, 1982.
Omirulleh et al., Plant Mol. Biol., 21(3):415-28, 1993.
PCT Appl. WO 94/09699
PCT Appl. WO 95/06128
Pedersen et al., Cancer Res., 54:120-123, 1994a.
Pedersen et al., Cancer Res., 54:4671-4675, 1994b.
Pepper et al., J. Biol. Chem., 267:20493-20496, 1992.
Potrykus et al., Mol. Gen. Genet., 199:183-188, 1985.
Potter et al., Proc. Nat. Acad. Sci. USA, 81:7161-7165, 1984.
Preiss et al., J. Biol. Chem., 268(33):24523-24526, 1993.
Remington's Pharmaceutical Sciences, 15^{th} ed., pages 1035-1038 and 1570-1580, Mack Publishing Company, Easton, Pa., 1980.
Ridgeway, In: Vectors: A survey of molecular cloning vectors and their uses, Rodriguez and Denhardt (Eds.), Stoneham: Butterworth, 467-492, 1988.
Rippe et al., Mol. Cell Biol., 10:689-695, 1990.
Robinson et al., Proc. Natl. Acad. Sci. USA, 86:8437-8441, 1989.
Roualt et al., Cell, 64:881-883, 1991.
Saksela and Rifkin, Annu. Rev. Cell Biol., 4:93-126, 1988.
Schmitt et al., Thromb. Haemost., 78:285-296, 1997.
Shetty and Idell, J. Biol. Chem., 275:13771-13779, 2000a.
Shetty and Idell, Am. J. Physiol.—Lung Cell. Mol. Physiol., 278, L148-156, 2000b.
Shetty and Idell, Arch. Biochem. Biophys., 356:265-279, 1998a.
Shetty and Idell, Am. J Physiol., 274:L871-L882, 1998b.
Shetty and Idell, Mol. Cell. Biochem., 199:189-200, 1999.
Shetty et al., Antisense Res. Dev., 5:307-314, 1995a.
Shetty et al., Am. J. Physiol., 268:L972-L982, 1995b.
Shetty et al., Mol. Cell. Biol., 17:1075-1083, 1997.
Sprengers and Kluft, Blood, 69:381-387, 1987.
Temin, In: Gene Transfer, Kucherlapati (Ed.), NY, Plenum Press, 149-188, 1986.
Thanos and Maniatis, Cell, 83:1091-1100, 1995.
Tur-Kaspa et al., Mol. Cell Biol., 6:716-718, 1986.
VandeBerg et al., J. Exp. Zool., 198:231-239, 1976.
VandeBerg, Isozymes Curr. Top Biol. Med. Res., 12:133-187, 1985.
Vassalli et al., J. Clin. Invest., 88:1067-1072, 1991.
Waltz et al., J. Clin. Invest., 91:1541-1552, 1993.
Wang et al., FEBS Letters, 353:138-142, 1994.
Wang et al., Proc. Natl. Acad. Sci. USA, 95:6296-6301, 1998.
Wong et al., Gene, 10:87-94, 1980.
Wu and Wu, Biochemistry, 27:887-892, 1988.
Wu and Wu, J. Biol. Chem., 262:4429-4432, 1987.
Xing et al., Cancer Res., 57:3585-3593, 1997.

1. A method of treating an inflammatory and/or neoplastic disease comprising administering to a subject a therapeutically effective amount of a phosphoglycerate kinase (PGK) peptide, polypeptide, protein, or a mutant or mimetic thereof, and inhibiting urokinase receptor (uPAR) activity or expression in the subject.

2. The method of claim 1, wherein the inflammatory disease is an inflammatory disease of the lung, thyroid, larynx, bladder, colon, esophagus, gastrointestine, gum, nasopharynx, or skin.

3. The method of claim 1, wherein the inflammatory disease is psoriasis, atopic dermatitis, nonspecific dermatitis, allergic contact dermatitis, primary irritant contact dermatitis, cutaneous basal cell carcinoma, cutaneous planocellular carcinoma, lameliar ichthyosis, epidemolytic keratosis, solar induced precancerous keratosis, benign keratosis, seborrheic dermatitis, keloids, dermatomyositis, angiogenesis-related skin disorders, and erythroderma.

4. The method of claim 1, further defined as a method to prevent sepsis.

5. The method of claim 1, further defined as a method to prevent inflammation after an injury.

6. The method of claim 5, wherein the injury is an immune tissue injury.

7. The method of claim 5, wherein the injury is a tissue injury resulting from exposure to environmental agents.

8. The method of claim 1, wherein the neoplastic disease is a cancer.

9. The method of claim 8, wherein the cancer is a premalignant cancer.

10. The method of claim 8, wherein the cancer is a malignant cancer.

11. The method of claim 8, wherein the cancer is a metastastic cancer.

12. The method of claim 8, wherein the cancer is a cancer of the lung, breast, head and neck, bladder, bone, bone marrow, brain, colon, esophagus, gastrointestine, gum, kidney, liver, nasopharynx, ovary, prostate, skin, stomach, testis, tongue, or uterus.

13. The method of claim 1, wherein a PGK polypeptide is administered to the subject.

14. The method of claim 13, wherein the PGK polypeptide comprises the sequence of SEQ ID NO:1.

15. The method of claim 13, wherein a PGK peptide, protein or protein mutant is administered to the patient.

16. The method of claim 1, wherein a PGK peptide, polypeptide, or protein mimetic is administered to a subject.

17. The method of claim 1, wherein the administering is carried out intravenously, intralesionally, percutaneously, subcutaneously, or by an aerosol.

18. The method of claim 1, wherein the PGK peptide, polypeptide, or protein, or mutant or mimetic reduces uPAR activity.

19. The method of claim 1, wherein the PGK peptide, polypeptide, or protein, or mutant or mimetic inhibits uPAR activity.

20. The method of claim 1, wherein the PGK peptide, polypeptide or protein, or mutant or mimetic reduces uPAR expression.

21. The method of claim 1, wherein the PGK peptide, polypeptide, or protein, or mutant or mimetic inhibits uPAR expression.

22. The method of claim 1, further comprising delivering an expression construct comprising a nucleic acid encoding a PGK peptide, polypeptide, or protein, or mutant or mimetic to a subject.

23. The method of claim 22, wherein the expression construct is a viral vector.

24. The method of claim 22, wherein the viral vector is an adenoviral vector, an adeno-associated viral vector, a herpesviral vector, a retroviral vector, a lentiviral vector, a vaccinia viral vector, or a polyoma vector.

25. The method of claim 1, wherein the subject is a mammal.

26. The method of claim 25, wherein the mammal is a human.

27. The method of claim 22, wherein the expression construct is delivered intravenously, intralesionally, percutaneously, subcutaneously, or by an aerosol.

28. The method of claim 1, further comprising gene therapy.

29. A method of screening a candidate substance for modulation of urokinase receptor (uPAR) expression or activity comprising:
a) providing a uPAR in a cell or a cell-free assay mixture;
b) contacting the uPAR with a candidate modulator substance; and
c) measuring the uPAR activity or expression,
wherein a decrease in the uPAR activity or expression in the presence of the candidate modulator as compared to the uPAR activity or expression in a cell or cell-free assay mixture not exposed to the candidate modulator indicates that the candidate phosphoglycerate kinase (PGK) substance has the ability to downregulate or inhibit the expression or activity of uPAR.

30. The method of claim 29, wherein the candidate substance is a peptide, polypeptide or protein.

31. The method of claim 29, wherein the candidate substance is a mutated peptide, polypeptide or protein.

32. The method of claim 29, wherein the candidate substance is a peptide, polypeptide or protein mimetic.

33. The method of claim 29, wherein the candidate substance is an organic small molecule.

34. The method of claim 29, wherein the candidate substance is an inorganic small molecule.

35. The method of claim 29, wherein the candidate substance is an expression construct.

36. The method of claim 29, wherein the candidate substance is a nucleic acid molecule.

37. The method of claim 29, wherein the cell is an inflammatory disease cell.

38. The method of claim 29, wherein the cell is a neoplastic disease cell.

39. The method of claim 29, wherein the cell is in a subject.

40. The method of claim 39, wherein the subject is a mammal.

41. The method of claim 29, wherein the cell is in vitro.

42. The method of claim 29, wherein measuring comprises Northern blotting.

43. The method of claim 29, wherein measuring comprises Western blotting.

44. The method of claim 29, further comprising the step of:
(d) manufacturing the candidate modulator substance.

45. The method of claim 44, further comprising the step of:
(e) administering a therapeutically effective amount of the candidate modulator substance to a subject.

46. The method of claim 45, wherein the subject is a human.

47. A method of promoting wound healing in a subject comprising administering to a subject a therapeutically effective amount of a phosphoglycerate kinase (PGK) peptide, polypeptide, protein, or a mutant or mimetic thereof, and inhibiting urokinase receptor (uPAR) activity or expression in the subject.

48. A method of preventing or decreasing scarring in a subject comprising administering to a subject a therapeutically effective amount of a phosphoglycerate kinase (PGK) peptide, polypeptide, protein, or a mutant or mimetic thereof; and inhibiting urokinase receptor (uPAR) activity or expression in the subject.

49. The method of claim 48, further defined as a method to prevent or decrease scarring after an injury.

50. A pharmaceutical composition comprising a phosphoglycerate kinase (PGK) peptide, polypeptide, protein, or a mutant, or a mimetic thereof.


SEQUENCE LISTING (2 sequences)

SEQ ID NO: 1 - DNA, 2338 bp, Homo sapiens
  CDS (70)..(1323)
agcgcacgtcggcagtcggctccctcgttgaccgaatcaccgacctctctccccagctgtatttccaaaatgtcgctttctaacaagctgacgctggacaagctggacgttaaagggaagcgggtcgttatgagagtcgacttcaatgttcctatgaagaacaaccagataacaaacaaccagaggattaaggctgctgtcccaagcatcaaattctgcttggacaatggagccaagtcggtagtccttatgagccacctaggccggcctgatggtgtgcccatgcctgacaagtactccttagagccagttgctgtagaactcaaatctctgctgggcaaggatgttctgttcttgaaggactgtgtaggcccagaagtggagaaagcctgtgccaacccagctgctgggtctgtcatcctgctggagaacctccgctttcatgtggaggaagaagggaagggaaaagatgcttctgggaacaaggttaaagccgagccagccaaaatagaagctttccgagcttcactttccaagctaggggatgtctatgtcaatgatgcttttggcactgctcacagagcccacagctccatggtaggagtcaatctgccacagaaggctggtgggtttttgatgaagaaggagctgaactactttgcaaaggccttggagagcccagagcgacccttcctggccatcctgggcggagctaaagttgcagacaagatccagctcatcaataatatgctggacaaagtcaatgagatgattattggtggtggaatggcttttaccttccttaaggtgctcaacaacatggagattggcacttctctgtttgatgaagagggagccaagattgtcaaagacctaatgtccaaagctgagaagaatggtgtgaagattaccttgcctgttgactttgtcactgctgacaagtttgatgagaatgccaagactggccaagccactgtggcttctggcatacctgctggctggatgggcttggactgtggtcctgaaagcagcaagaagtatgctgaggctgtcactcgggctaagcagattgtgtggaatggtcctgtgggggtatttgaatgggaagcttttgcccggggaaccaaagctctcatggatgaggtggtgaaagccacttctaggggctgcatcaccatcataggtggtggagacactgccacttgctgtgccaaatggaacacggaggataaagtcagccatgtgagcactgggggtggtgccagtttggagctcctggaaggtaaagtccttcctggggtggatgctctcagcaatatttagtactttcctgccttttagttcctgtgcacagcccctaagtcaacttagcattttctgcatctccacttggcattagctaaaaccttccatgtcaagattcagctagtggccaagagatgcagtgccaggaacccttaaacagttgcacagcatctcagctcatcttcactgcaccctggatttgcatacattcttcaagatcccatttgaattttttagtgactaaaccattgtgcattctagagtgcatatatttatattttgcctgttaaaaagaaagtgagcagtgttagcttagttctcttttgatgtaggttattatgattagctttgtcactgtttcactactcagcatggaaacaagatgaaattccatttgtaggtagtgagacaaaattgatgatccattaagtaaacaataaaagtgtccattgaaaccgtgatttttttttttttcctgtcatactttgttaggaagggtgagaatagaatcttgaggaacggatcagatgtctatattgctgaatgcaagaagtggggcagcagcagtggagagatgggacaattagataaatgtccattctttatcaagggcctactttatggcagacattgtgctagtgcttttattctaacttttatttttatcagttacacatgatcataatttaaaaagtcaaggcttataacaaaaaagccccagcccattcctcccattcaagattcccactccccagaggtgaccactttcaactcttgagtttttcaggtatatacctccatgtttctaagtaatatgcttatattgttcacttcctttttttttattttttaaagaaatctatttcataccatggaggaaggctctgttccacatatatttccacttcttcattctctcggtatagttttgtcacaattatagattagatcaaaagtctacataactaatacagctgagctatgtagtatgctatgattaaatttacttatgtaaaaaaaaaaaaaaaaaaaa

SEQ ID NO: 2 - protein, 417 aa, Homo sapiens
MSLSNKLTLDKLDVKGKRVVMRVDFNVPMKNNQITNNQRIKAAVPSIKFCLDNGAKSVVLMSHLGRPDGVPMPDKYSLEPVAVELKSLLGKDVLFLKDCVGPEVEKACANPAAGSVILLENLRFHVEEEGKGKDASGNKVKAEPAKIEAFRASLSKLGDVYVNDAFGTAHRAHSSMVGVNLPQKAGGFLMKKELNYFAKALESPERPFLAILGGAKVADKIQLINNMLDKVNEMIIGGGMAFTFLKVLNNMEIGTSLFDEEGAKIVKDLMSKAEKNGVKITLPVDFVTADKFDENAKTGQATVASGIPAGWMGLDCGPESSKKYAEAVTRAKQIVWNGPVGVFEWEAFARGTKALMDEVVKATSRGCITIIGGGDTATCCAKWNTEDKVSHVSTGGGASLELLEGKVLPGVDALSNI